SEQUENCE LISTING (56 sequences)

SEQ ID NO: 1 - DNA, 14 bp, Artificial Sequence
  Description of Artificial Sequence Synthetic oligonucleotide
tggtgcatggttgt

SEQ ID NO: 2 - DNA, 14 bp, Artificial Sequence
  Description of Artificial Sequence Synthetic oligonucleotide
tgctgcatggctgt

SEQ ID NO: 3 - DNA, 24 bp, Artificial Sequence
  Description of Artificial Sequence Synthetic probe
aggtggtgcatggttgtcgtcagc

SEQ ID NO: 4 - DNA, 27 bp, Artificial Sequence
  Description of Artificial Sequence Synthetic probe
acaggtgctgcatggctgtcgtcagct

SEQ ID NO: 5 - DNA, 22 bp, Artificial Sequence
  Description of Artificial Sequence Synthetic primer
tggagcatgtggtttaattcga

SEQ ID NO: 6 - DNA, 19 bp, Artificial Sequence
  Description of Artificial Sequence Synthetic primer
tgcgggacttaacccaaca

SEQ ID NO: 7 - DNA, 23 bp, Artificial Sequence
  Description of Artificial Sequence Synthetic probe
cacgagctgacgacarccatgca

SEQ ID NO: 8 - DNA, 31 bp, Artificial Sequence
  Description of Artificial Sequence Synthetic probe
cctttgacaactctagagatagagccttccc

SEQ ID NO: 9 - DNA, 19 bp, Artificial Sequence
  Description of Artificial Sequence Synthetic primer
gyggcgnacgggtgagtaa

SEQ ID NO: 10 - DNA, 17 bp, Artificial Sequence
  Description of Artificial Sequence Synthetic primer
ttaccyyaccaactagc

SEQ ID NO: 11 - DNA, 21 bp, Artificial Sequence
  Description of Artificial Sequence Synthetic primer
ccagactcctacgggaggcag

SEQ ID NO: 12 - DNA, 19 bp, Artificial Sequence
  Description of Artificial Sequence Synthetic primer
cgtattaccgcggctgctg

SEQ ID NO: 13 - DNA, 22 bp, Artificial Sequence
  Description of Artificial Sequence Synthetic primer
tggagcatgtggtttaattcga

SEQ ID NO: 14 - DNA, 19 bp, Artificial Sequence
  Description of Artificial Sequence Synthetic primer
agctgacgacarccatgca

SEQ ID NO: 15 - DNA, 30 bp, Artificial Sequence
  Description of Artificial Sequence Synthetic probe
cctctgacaaccctagagatagggcttctc

SEQ ID NO: 16 - DNA, 30 bp, Artificial Sequence
  Description of Artificial Sequence Synthetic probe
cacagaatttggcagagatgctaaagtgcc

SEQ ID NO: 17 - DNA, 33 bp, Artificial Sequence
  Description of Artificial Sequence Synthetic probe
cgaactttctagagatagattggtgcttcggaa

SEQ ID NO: 18 - DNA, 30 bp, Artificial Sequence
  Description of Artificial Sequence Synthetic probe
aagggaaagctctgtctccagagtggtcaa

SEQ ID NO: 19 - DNA, 30 bp, Artificial Sequence
  Description of Artificial Sequence Synthetic probe
tgctccgaagagaaagcctatctctaggcc

SEQ ID NO: 20 - DNA, 36 bp, Staphylococcus epidermidis
aaaactctatctctagaggggctagaggatgtcaag

SEQ ID NO: 21 - DNA, 35 bp, Artificial Sequence
  Description of Artificial Sequence Synthetic probe
tcacctctgtcccgaaggaaaactctatctctaga

SEQ ID NO: 22 - DNA, 33 bp, Artificial Sequence
  Description of Artificial Sequence Synthetic probe
acattctcatctctgaaaacttccgtggatgtc

SEQ ID NO: 23 - DNA, 33 bp, Artificial Sequence
  Description of Artificial Sequence Synthetic probe
aaggcacaagctcatctctgagctcttcttagg

SEQ ID NO: 24 - DNA, 25 bp, Artificial Sequence
  Description of Artificial Sequence Synthetic probe
cactcctccgtctccggaggattcc

SEQ ID NO: 25 - DNA, 47 bp, Acinetobacter sp.
ttcgggaacttacatacaggtgctgcatggctgtcgtcagctcgtgt

SEQ ID NO: 26 - DNA, 47 bp, Pseudomonas sp.
ttcgggaactctgacacaggtgctgcatggttgtcgtcagctcgtgt

SEQ ID NO: 27 - DNA, 47 bp, Aeromonas caviae
ttcgggaatcagaacacaggtgctgcatggctgtcgtcagctcgtgt

SEQ ID NO: 28 - DNA, 47 bp, Aeromonas hydrophila; identical to SEQ ID NO: 27

SEQ ID NO: 29 - DNA, 47 bp, Klebsiella pneumoniae
ttcgggaactgtgagacaggtgctgcatggctgtcgtcagctcgtgt

SEQ ID NO: 30 - DNA, 47 bp, Serratia marcescens
ttcgggaactctgagacaggtgctgcatggctgtcgtcagctcgtgt

SEQ ID NO: 31 - DNA, 47 bp, Enterobacter sp.; identical to SEQ ID NO: 30

SEQ ID NO: 32 - DNA, 47 bp, Escherichia coli
ttcgggaaccgtgagacaggtgctgcatggctgtcgtcagctcgtgt

SEQ ID NO: 33 - DNA, 47 bp, Citrobacter freundii
  modified_base (32)..(32): a, c, t, g, unknown or other
ttcgggaactctgagacaggtgctgcatggcngtcgtcngctcgtgt

SEQ ID NO: 34 - DNA, 47 bp, Salmonella sp.; identical to SEQ ID NO: 29

SEQ ID NO: 35 - DNA, 47 bp, Yersinia pestis; identical to SEQ ID NO: 29

SEQ ID NO: 36 - DNA, 47 bp, Morganella morganii; identical to SEQ ID NO: 30

SEQ ID NO: 37 - DNA, 47 bp, Proteus sp.
ttcgggaacgctgagacaggtgctgcatggctgtcgtcagctcgtgt

SEQ ID NO: 38 - DNA, 47 bp, Haemophilus influenzae
ttcgggaacttagagacaggtgctgcatggctgtcgtcagctcgtgt

SEQ ID NO: 39 - DNA, 47 bp, Legionella pneumophila
ttcgggaacactgatacaggtgctgcatggctgtcgtcagctcgtgt

SEQ ID NO: 40 - DNA, 47 bp, Neisseria meningitidis
ttcgggagccgtaacacaggtgctgcatggctgtcgtcagctcgtgt

SEQ ID NO: 41 - DNA, 47 bp, Stenotrophomonas sp.
ttcgggaacgcgaacacaggtgctgcatggctgtcgtcagctcgtgt

SEQ ID NO: 42 - DNA, 44 bp, Francisella tularensis
tcggaacgcagtgacagtgctgcacggctgtcgtcagctcgtgt

SEQ ID NO: 43 - DNA, 49 bp, Brucella sp.
gttcggctggatcggagacaggtgctgcatggctgtcgtcagctcgtgt

SEQ ID NO: 44 - DNA, 49 bp, Mycobacterium sp.
tcccttgtggcctgtgtgcaggtggtgcatggctgtcgtcagctcgtgt

SEQ ID NO: 45 - DNA, 49 bp, Campylobacter jejuni
cttgctagaacttagagacaggtgctgcacggctgtcgtcagctcgtgt

SEQ ID NO: 46 - DNA, 46 bp, Mycoplasma pneumoniae
aggttaaccgagtgacaggtggtgcatggttgtcgtcagctcgtgt

SEQ ID NO: 47 - DNA, 49 bp, Propionibacterium sp.
tcttttggggttggttcacaggtggtgcatggctgtcgtcagctcgtgt

SEQ ID NO: 48 - DNA, 49 bp, Corynebacterium sp.
  modified_base (38)..(38): a, c, t, g, unknown or other
tcccttgtggctcacatacaggtggtgcatggttgtcntcagctcgtgt

SEQ ID NO: 49 - DNA, 48 bp, Enterococcus sp.
cttcgggggcaaagtgacaggtggtgcatggttgtcgtcagctcgtgt

SEQ ID NO: 50 - DNA, 49 bp, Staphylococcus aureus
cttcgggggacaaagtgacaggtggtgcatggttgtcgtcagctcgtgt

SEQ ID NO: 51 - DNA, 49 bp, Bacillus sp.
cttatgggacagcggtgacaggtggtgcatggttgtcgtcagctcgtgt

SEQ ID NO: 52 - DNA, 49 bp, Streptococcus pneumoniae
  modified_base (1)..(1): a, c, t, g, unknown or other
ncttcgggacagaggngacaggtggngcatngtngtcgtcagctcgtgt

SEQ ID NO: 53 - DNA, 49 bp, Streptococcus sp.
acttcggtacatcggtgacaggtggtgcatggttgtcgtcagctcgtgt

SEQ ID NO: 54 - DNA, 49 bp, Clostridium sp.
ccttcggggacagggagacaggtggtgcatggttgtcgtcagctcgtgt

SEQ ID NO: 55 - DNA, 49 bp, Peptostreptococcus sp.
tcttcggagactgctaaacaggtggtgcatggttgtcgtcagctcgtgt

SEQ ID NO: 56 - DNA, 28 bp, Artificial Sequence
  Description of Artificial Sequence Synthetic probe
tctccggaggattccgcacatgtcaaaa

(19) United States
(12) Patent Application Publication
(10) Pub. No.: US 2013/0217588 A1
(43) Pub. Date: Aug. 22, 2013

(54) Methods for Identifying Eubacteria

(71) Applicants: Samuel Yang, Columbia, MD (US); Richard Rothman, Cockeysville, MD (US); Charlotte Gaydos, Bel Air, MD (US)

(72) Inventors: Samuel Yang, Columbia, MD (US); Richard Rothman, Cockeysville, MD (US); Charlotte Gaydos, Bel Air, MD (US)

(73) Assignee: THE JOHNS HOPKINS UNIVERSITY, Baltimore, MD (US)

(21) Appl. No.: 12/998,621

(22) PCT Filed: Jan. 21, 2009
(86) PCT No.: PCT/US2009/031583

§ 371 Date: Dec. 15, 2011

(30) Foreign Application Priority Data
Jan. 18, 2008 (US) 61011522
Jan. 18, 2008 (US) 61011529
Mar. 6, 2008 (US) 61068345

(51) Int. Cl. G01N 21/64; C40B 30/04; C12Q 1/68

(52) U.S. Cl. 506/9; 435/6.11

(57) ABSTRACT

This invention relates, e.g., to methods for detecting a aubacterium, determining if the eubacterium is Gram-positive or Gram-negative, and determining the species of the eubacterium in a sample.

This application claims the benefit of the filing date of provisional patent applications 61/011,522, filed Jan. 18, 2008; 61/011,529, filed Jan. 18, 2008; and 61/068,345, filed Mar. 6, 2008, all of which are incorporated by reference in their entireties herein.

This application was made with U.S. government support, including Mid-Atlantic Regional Centre of Excellence grant-NIH (AI-02-031) from NIAID. The U.S. government thus has certain rights in the invention.

### BACKGROUND INFORMATION

Rapid and accurate diagnostic tools are critical for infectious disease surveillance and early diagnosis of disease. A simple platform which could provide broad-based screening and specific pathogen identification would accordingly be invaluable, both for more rapid diagnosis of commonly encountered infections seen in clinical settings and timely recognition of emerging and biothreat (BT) outbreaks.

For example, septic arthritis (SA) is a rheumatologic emergency associated with significant morbidity and mortality. Delayed or inadequate treatment of SA can lead to irreversible joint destruction and disability. The diagnosis of SA in the acute-care setting is challenging because of the relatively poor sensitivity and specificity of clinical examination findings, as well as lack of a rapid reliable diagnostic assay. Further, overreliance on conventional laboratory tests for synovial fluid analysis is hindered by the relatively poor performance characteristics of these methods. In particular, the sensitivity of Gram stain has been reported in the range of 29% to 50% and the sensitivity of culture may only be 82%. Lack of a rapid and accurate diagnostic tool results in acute-care clinicians often choosing the conservative approach of hospital admission and empiric broad spectrum antibiotics for patients with suspected SA. The benefits of this management strategy may be offset, however, by added costs and potential iatrogenic complications associated with unnecessary treatment and hospitalizations, as well as increased rates of antimicrobial resistance. A sensitive, specific diagnostic assay, which allows for rapid definitive diagnosis of SA and directed therapeutic intervention, would thus be invaluable in the acute care setting.

Furthermore, because the emergency department (ED) is now recognized by many in the infectious disease and public health community as the frontline for early identification of biothreat and emerging infections, the capacity for rapid and accurate diagnosis of infectious disease outbreaks in the ED is critical both for individual patient care and initiation of timely public health countermeasures. Unfortunately, rapid recognition of infections caused by new or unexpected pathogens, which frequently present with nonspecific clinical syndromes, is extremely difficult, and reliance on either the astute clinician or syndromic surveillance methods may prove inadequate. From the ED standpoint, current traditional hospital diagnostic assays, which are culture based, have limited to no utility in ED settings in the event of a suspected outbreak, due to prolonged wait times required for growth. Furthermore, although specialized assays are available at centralized public health laboratories, the utility of such tools for ED care is limited, due to inherent delays associated with transporting specimens to outside laboratories as well as design of the assays themselves, which are pathogen specific. A diagnostic platform which has the capacity for both rapid broad-based detection of any bacterial agent, as well as specific pathogen identification would thus be highly desirable for acute care setting use. Applicability in the ED includes not only early detection of biothreat or emerging pathogens, but also potential for everyday use in expediting diagnosis of systemic eubacterial infections.

### DESCRIPTION OF THE DRAWINGS

FIG. 1 is a cartoon showing the approximate locations of some of the primers and probes used in a method of the invention.

FIG. 2 shows the difference plot of all the Cat A BT bacterial organisms from Example III and their surrogates analyzed and grouped as three different primer sets (V1, V3 and V6). The grouping code and the analysis sets are marked. BAAN—B. anthracis, BACE—B. cereus, YEPE—Y. pestis, YEPS—Y. pseudotuberculosis, FRTU—F. tularensis, FRPH—F. phylomiragia.

### DESCRIPTION OF THE INVENTION

The inventors previously reported a probe-based real-time polymerase chain reaction (RT-PCR) assay, which utilizes conserved and variable 16S rRNA gene sequences for initial broad-based eubacteria detection, and subsequent or simultaneous identification of specific bacterial agents (See, e.g., Yang et al. (2002) J Clin Microbiol. 40, 3449-5411 and U.S. Patent application 2004/0235010, both of which are incorporated by reference herein in their entireties). The assay for detecting the presence of eubacteria relies on a probe for a conserved sequence within the 16S rRNA gene, which is sometimes referred to herein as a “Uniprobe.” The assay for determining the species of eubacterium relies on a probe to a specific hypervariable region within the 16S rRNA gene. Such a probe is sometimes referred to herein as a “species-specific” probe.

The inventors disclose herein two significant improvements over their previously described “Uniprobe/species-specific” assay method, as well as a new assay method based on melt curve analysis. These methods can be performed independently, or can be combined with one another.

One improvement over the previously described assay method is to add to the Uniprobe and the species-specific probe a third probe, which can detect whether a eubacterium is Gram-positive or Gram-negative. Gram-positive or Gram-negative specific probes are designed from a conserved region of the bacterial 16S rRNA. An investigator can first perform the Gram typing assay method, and based on the results, can select species-specific probes to screen for species that are either Gram-positive or Gram-negative in a subsequent step. This preliminary screening step reduces the total number of species-specific probes which must be used in the assay. Furthermore, in cases in which a detected bacterium is not identified by a panel of species-specific probes, a Gram-typing test can help a clinician select a suitable antibiotic for treatment, by providing additional characterization of the detected bacterium. Gram-typing tests can also provide an additional confirmation of the etiologic agent identified by a species-specific probe. As proof-of-principle, the inventors used the improved assay method in the Examples herein to test for the presence of six eubacteria that are diagnostic of septic arthritis (SA), and whose presence can distinguish subjects having this diagnosis from those with clinically indistinguishable symptoms from other causes of joint inflammation. The platform demonstrated high analytical sensitivity with a limit of detection (LOD) of 10¹-10² CFU/ml with a panel of SA-related organisms. Gram-typing and pathogen-specific probes correctly identified their respective targets in a mock test panel of 36 common clinically relevant pathogens. One hundred twenty one clinical synovial fluid samples from patients presenting with suspected acute SA were tested. Sensitivity and specificity of the assay were 95% and 97%, respectively, versus synovial culture results. Gram-typing probes correctly identified 100% of eubacterial positive samples as to Gram-positive or Gram-negative, and pathogen-specific probes correctly identified the etiologic agent at the species level in 16/20 eubacterial positive samples.

Advantages of this assay method include 1) high sensitivity and specificity; 2) capacity for early pathogen characterization; and 3) rapidity coupled with simple sample processing and identification. The total assay time from sample collection to result is less than 3 hours. This compares with 1-2 days minimum for routine culture (longer for fastidious bugs). An assay with high detection sensitivity for SA would be particularly desirable in an acute care setting, because reliable negative results would allow major changes in clinical management. For example, patients who are being hospitalized solely for ruling out SA could be safely discharged without having to wait for culture results.

A second improvement over the previously described assay method is a novel procedure for preparing DNA samples for analysis by assays requiring relatively large quantities of DNA that is free of contaminating exogenous eubacterial DNA. Such contaminating DNA can be pronounced, for example, in broad-based 16S rRNA eubacterial assays. Incorporation of a combination of chaotropic, thermal and enzymatic inductions of cell lysis in the sample processing protocol, involving a limited number of transfer steps, allows one to achieve high detection sensitivity via effective release of microbial DNA, even from difficult-to-lyse cell walls of Gram-positive organisms. The procedure involves steps to lyse cells and to digest protein; no further isolation (purification) of the DNA is required. Notably, in Example I herein, in which the DNA samples are prepared by this procedure, the inventors show that only one PCR-negative, culture-positive sample occurred in a probe-based RT-PCR assay of the invention; and that culture may have resulted from a laboratory contamination event, since bacterial growth from this sample was only detected in culture broth, and not by conventional plating. This procedure for DNA preparation can be used with any assay in which a large amount of non-contaminated host cellular DNA is required. For example, the procedure can be used to prepare DNA for the previously disclosed Uniprobe/species-specific probe assay method. The DNA sample preparation procedure can also be used advantageously in conjunction with the improved probe-based real-time PCR assay targeting assay described herein, in which Gram typing is included; in the melt curve assay method described below; and in other assay methods that will be evident to a skilled worker.

Advantages of this procedure include, e.g., that it effectively releases microbial DNA content from all bacterial cells, achieves excellent bacterial DNA recovery (which is often a problem in procedures that involve extraction and purification steps), minimizes transferring steps and thus minimizes contamination with background bacterial DNA, can accommodate large sample volumes for processing, and can be easily adapted for automation with enhanced throughput. The procedure allows for a limit of detection, in combination with the eubacterial PCR assay described herein, of as little as 1 CFU per ml of sample. The currently processing time is less than 1 hour. The described procedure for DNA preparation can increase the limit of detection for eubacterial DNA over other, conventional methods by as much as 1,000-fold.

A third assay method for identifying species of eubacteria which is described herein involves PCR amplification of segments of 16S rRNA, which are hypervariable regions flanked by sequences that are highly conserved in eubacteria. The amplified DNAs are then analyzed by high resolution melt curve profile analysis (sometimes referred to herein as the “melt curve” method). A database of melting profile “signatures,” each unique and specific to a bacterial species, is created for identifying an unknown organism. Genotyping based on melting analysis exploits differences in melt curves generated based on sequence variations. Despite the ability to discriminate single nucleotide variation, DNA with entirely different sequences may occasionally result in similar melting profiles. To overcome this limitation, multiple genetic target sites are queried to enhance the discriminatory power of melting analysis. The amplicons in this method are small, e.g., between about 25 and 75 bp. At least three amplicons are examined by this melt curve method to detect and characterize each bacterium.

Advantages of this melt curve assay method include that the method is rapid, accurate and inexpensive. Furthermore, the small size of the amplicons, and the fact that at least three hypervariable regions of the 16S rRNA gene are assayed, allows one to differentiate closely related species. For example, the small size of the amplicons allows an investigator to generate melt profiles that are not compromised by interference resulting from nucleotide polymorphisms or other variations between bacterial strains of the same species. The variability of melt curves is more pronounced with shorter than longer sequences, allowing one to make more accurate distinctions among species. Another advantage of this assay method is that, unlike probe based approaches to amplicon analysis (e.g. TaqMan PCR, or microarray), melt curve analysis can characterize PCR products without a priori knowledge of anticipated organisms. Accordingly, a reference database of melt curve signatures can be expanded to include a wide range of commonly encountered bacterial pathogens and non-bacterial pathogens. If a melt curve profile from a positive amplification reaction does not match existing signatures in the database, it may signify presence of an uncommon, mutant, or emerging pathogen. This approach offers a simple work flow with total turnaround time of 2 hours (from sample collection to species identification) and obviates need for laborious post PCR procedures or amplicon analysis based on sequencing. Due to the ease of integrating melt analysis, this approach has the potential to be used as a point-of-care test, and may be feasible in resource-deficient clinical settings. If desired, an investigator can first determine if a sample comprises eubacteria by a Uniprobe method, with or without the addition of Gram typing, and then can perform the melt curve method on samples that are positive for eubacteria.

An advantage of any of the methods of the invention is that they can readily be adapted to high throughput format, using automated (e.g., robotic) systems, which allow many measurements to be carried out simultaneously. Furthermore, the methods can be miniaturized.

Methods of the invention can be used in a variety of applications, e.g., to characterize bacteria present in clinical samples (to determine if a subject is bacteremic), to determine whether biothreat (BT) bacteria are present in a sample, or in other applications which will be evident to a skilled worker.

One aspect of the present invention is a set of oligonucleotides for distinguishing Gram-positive eubacteria from Gram-negative eubacteria, wherein

(a) a “first” oligonucleotide, which is specific for Gram-positive eubacteria, consists of the sequence TGGTGCATGGTTGT (SEQ ID NO:1);
or a variant thereof in which 1 or 2 of the residues are substituted with other nucleotides, provided that the G and T residues that are indicated with bold underlining are not altered;
or a variant of either the oligonucleotide of SEQ ID NO:1 or of the substituted variant thereof, which has up to 5 additional nucleotides at its 5′ end from the Propionibacter rRNA gene sequence shown in Table 7 and/or up to 13 additional nucleotides at its 3′ end from the Propionibacter rRNA gene sequence shown in Table 7;

and

(b) a “second” oligonucleotide, which is specific for Gram-negative eubacteria, consists of the sequence TGCTGCATGGCTGT (SEQ ID NO:2);
or a variant thereof in which 1 or 2 of the residues are substituted with other nucleotides, provided that the two C residues that are indicated with bold underlining are not altered;
or a variant of either the oligonucleotide of SEQ ID NO:2 or of the substituted variant thereof, which has up to 4 additional nucleotides at its 5′ end from the Acinetobacter rRNA gene sequence shown in Table 7 and/or up to 13 additional nucleotides at its 3′ end from the Acinetobacter rRNA gene sequence shown in Table 7.

For example, the set of oligonucleotides can comprise (a) a “first” oligonucleotide, which is specific for Gram-positive eubacteria, which consists of the sequence AGGTGGTGCATGGTTGTCGTCAGC (SEQ ID NO:3), or a variant thereof in which 1-3 of the residues are substituted with other nucleotides, provided that the G and T residues that are indicated with bold underlining are not altered; and (b) a “second” oligonucleotide, which is specific for Gram-negative eubacteria, which consists of the sequence ACAGGTGCTGCATGGCTGTCGTCAGCT (SEQ ID NO:4), or a variant thereof in which 1-3 of the residues are substituted with other nucleotides, provided that the two C residues that are indicated with bold underlining are not altered. The oligonucleotide represented by SEQ ID NO:3 is sometimes referred to herein as the Universal Gram positive probe. The oligonucleotide represented by SEQ ID NO:4 is sometimes referred to herein as the Universal Gram negative probe.

Another aspect of the invention is a method that is specifically designed to use the above sets of oligonucleotides; the oligonucleotides serve as probes for determining whether a eubacterium in a sample is Gram-positive or Gram-negative. The “first” oligonucleotides described above are specific for Gram-positive eubacteria, and the “second” oligonucleotides described above are specific for Gram-negative eubacteria. In this method, template DNA in a sample, which may comprise (is suspected of comprising) template DNA of the eubacterium, is amplified using a real-time polymerase chain reaction (RT-PCR). The RT-PCR employs primers and at least two fluorogenic probes, which are specific for either Gram-positive or Gram-negative eubacteria.

The primers amplify a segment of the S. aureus 16S rRNA that comprises the sequence, of SEQ ID NO:3 (which is present in Gram-positive eubacteria, including S. aureus) or, in Gram-negative eubacteria, amplify a segment of the rRNA gene that comprises SEQ ID NO:4 (which is present in Gram-negative eubacteria). Because the PCR-amplified DNA in a test sample is double-stranded, a probe of the invention can bind to one of the two DNA strands of the double-stranded DNA, and is completely complementary to the other strand. Which of the two strands is being described is not always indicated in the discussion herein; but it will be evident to a skilled worker whether a given probe binds to one of the DNA strands of an amplicon, or to its complete complement. In one embodiment of the invention, the amplified segment (the amplicon) is about 160 bp in length. The first of the at least two fluorogenic probes has the sequence of (or is completely complementary to) one of the “first” oligonucleotides described above; and the second of the at least two fluorogenic probes has the sequence of (or is completely complementary to) one of the “second” oligonucleotides described above. Each of the two fluorogenic probes in the RT-PCR reaction comprises a reporter dye and a quencher dye; the reporter dyes of the two probes have non-overlapping emission spectra. Fluorescence emissions of the reporter dyes are monitored. If emissions are detected that are characteristic of the reporter dye of the “first” probe, this indicates that the eubacterium being assayed is Gram-positive. If emissions are detected that are characteristic of the reporter dye of the “second” probe, this indicates that the eubacterium being assayed is Gram-negative.

Another aspect of the invention is an assay method for detecting a eubacterium, determining if the eubacterium is Gram-positive or Gram-negative, and determining the species of the eubacterium (genotyping the eubacterium) in a sample. In this assay method, template DNA in a sample, which may comprise (is suspected of comprising) template DNA of the eubacterium, is amplified using a RT-PCR reaction, which employs primers and at least one fluorogenic probe.

The primers amplify a segment of a S. aureus 16S rRNA gene comprising a first conserved region (which is emblematic of eubacteria), a second conserved region (which is diagnostic for (present in) either Gram-positive or Gram-negative bacteria), and a first divergent region, if a S. aureus 16S rRNA gene is present in the PCR reaction. Phrases such as “detecting a eubacterium” are not meant to exclude samples or determinations (detection attempts) wherein no analyte is contained or detected. In a general sense, this invention involves a method to determine whether an analyte (a eubacterium) is present in a sample, irrespective of whether it is detected or not. The discussion in the remainder of this paragraph refers to sequences of one strand of the 16S rRNA double-stranded rRNA gene. A skilled worker will recognize that the other strand of the DNA comprises the complete complement of these sequences. The first conserved region comprises at least 18 contiguous nucleotides which are at least 80% identical among at least 10 eubacterial species. The second conserved region comprises the sequence represented by SEQ ID NO:3 or SEQ ID NO:4 (and, on the other strand of the DNA molecule, the complete complement of SEQ ID NO:3 or SEQ ID NO:4). The first divergent region comprises at least 10 contiguous nucleotides and differs by at least 3 nucleotides from a second divergent region found in the Bradyrhizobium japonicum 16S rRNA gene. In one embodiment of the invention, the PCR primers used to amplify this segment of eubacterial 16S rRNA are the forward primer 5′TGGAGCATGTGGTTTAATTCGA3′ (SEQ ID NO:5), which extends from position 890-912, and is sometimes referred to herein as P890F; and the reverse primer 5′TGCGGGACTTAACCCAACA3′ (SEQ ID NO:6), which extends from 1033-1051, and is sometimes referred to herein as P1033R. The nucleotide positions of these primers are based on S. aureus sequences (AF015929).

Each of the fluorogenic probes comprises a reporter dye and a quencher dye. A first fluorogenic probe is complementary to (and hybridizes specifically to) the first conserved region; the second fluorogenic probe is complementary to (and hybridizes specifically to) one of the “first” (Gram-positive specific) oligonucleotides described above; the third fluorogenic probe is complementary to (and hybridizes specifically to) one of the “second” (Gram-negative) oligonucleotides described above; and the fourth of the fluorogenic probes is complementary to (and hybridizes to) a third divergent region of a first species of eubacteria. In one embodiment of the invention, the first fluorogenic probe, which hybridizes specifically to the first conserved region, is 5′CACGAGCTGACGACARCCATGCA3′ (SEQ ID NO:7); it is sometimes referred to herein as the Universal Probe or the Uniprobe. This probe is the reverse complement of nucleotides 1002 to 1024 of the 16S rRNA gene (numbering according to the S. aureus sequence AFO15929). In one embodiment of the invention, the fourth flourogenic probe hybridizes to the divergent region of S. aureus which extends from position 945-978 (numbering according to S. aureus sequence AFO15929). This probe has the sequence 5′CCTTTGACAACTC TAGAGATAGAGCCTTCCC3′ (SEQ ID NO:8), and is sometimes referred to herein as the SAProbe.

Between one and four of the fluorogenic probes are present in each RT-PCR. If two or more probers are present in a single RT-PCR, the reporter dyes of the two or more dyes must have non-overlapping emissions spectra, to allow for the multiple probes to be distinguished. If only one probe is present in an RT-PCR, the reporter dyes may have identical, overlapping, or non-overlapping emissions spectra. Fluorescence emissions of the reporter dyes are monitored. The detection of emissions characteristic of the reporter dye of the first probe indicate that a eubacterium is present in the sample. The detection of emissions characteristic of the reporter dye of the second probe or the third probe indicate that the eubacterium in the sample is Gram-positive or Gram negative, respectively. The detection of emissions characteristic of the reporter dye of the fourth probe indicates that the first species of eubacteria is present in the sample.

Another aspect of the invention is an assay method for determining the species of a eubacterium in a sample, which comprises:

(a) performing three different PCRs (e.g., RT-PCRs), using three aliquots of a sample which may contain template DNA of a eubacterium, wherein the size of the amplicons generated in each reaction is less than about 100 bp, e.g., less than 75 bp, e.g., between about 25 and about 75 bp. The paper, Chakravorty et al. (2007) (J Microbiol Methods 69, 330-339), which is incorporated by reference in its entirely herein, identified nine hypervariable regions of the eubacterial 16S rRNA gene, each of which is flanked by highly conserved regions. See also Clarridge et al. (2004) Clin Microbial Rev 17, 840-862. The primers for each of the three PCRs of this method of the invention are designed to amplify one of hypervariable regions V1, V3 or V6. Suitable primers can be designed and prepared for each of the hypervariable regions by a skilled worker. The positions of the following primers are based on the S. aureus sequence (AF015929). The primers in the first PCR reaction, which amplify a sequence within hypervariable region V1, can be, for example, V1-F: 5′-GYGGCGNACGGGTGAGTAA-3′ (SEQ ID NO:9) and V1-R: 5′-TTACCYYACCAACTAGC-3′ (SEQ ID NO:10), which amplify a sequence of about 162 bp. The primers in the second PCR reaction, which amplify a sequence within hypervariable region V3, can be, for example, V3-F: 5′-CCAGACTCCTACGGGAGGCAG-3′ (SEQ ID NO:11) and V3-R: 5′-CGTATTACCGCGGCTGCTG-3′ (SEQ ID NO:12), which amplify a sequence of about 205 bp. The primers in the third PCR reaction, which amplify a sequence within hypervariable region V6 can be, for example, V6-F: 5′-TGGAGCATGTGGTTTAATTCGA-3′ (SEQ ID NO:13) and V6-R: 5′-AGCTGACGACARCCATGCA-3′ (SEQ ID NO:14), which amplify a sequence of about 131 bp;

(b) subjecting each of the three PCR-amplified DNA preparations (amplicons) to High Resolution Melting Analysis (HRMA), to generate a melt curve for each of the three amplified DNAs, and a composite melt profile taking into account all three of the melt curves; and

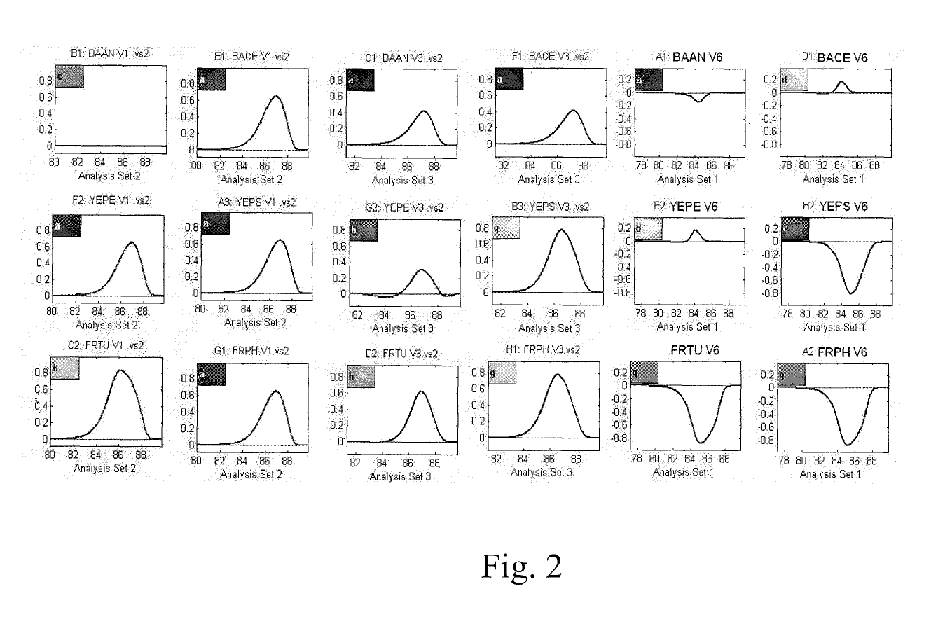

(c) comparing the melt profiles from the three PCR-amplified DNA preparations to a reference database of melt profiles for the three regions of a large number (e.g., at least 30, 40, 50, 60, 70 or more) bacterial species, for example as indicated in Table 8 and FIG. 2.

If the melt profile from the three regions for a sample corresponds to (e.g., is the same as) the melt profile from the three regions of a known bacterial species, this indicates that that bacterial species is present in the sample.

The PCR reactions to generate the amplicons to he subjected to melt analysis can be real-time PCRs, or non-real-time PCRs.

For any of the methods of the invention, the DNA can be prepared by a procedure devised by the inventors, which allows for the preparation of large enough quantities of DNA to function in the method, yet the DNA is free of contaminating exogenous eubacterial DNA. In this method, DNA is prepared by (a) concentrating the sample, for example centrifuging the sample under conditions effective to pellet cells in the sample, (b) resuspending the concentrated (pelleted) cells in a suitable diluent, such as molecular grade water, which has been previously decontaminated from bacterial DNA with ultra-filtration, (c) incubating the resuspended cells with Lysostaphin and Proteinase K, under conditions effective to lyse a significant number (e.g., at least 10%) of the cells and to degrade proteins in the cells, and (d) subjecting the enzyme treated samples to one or more cycles of freezing and thawing, or to another mechanical method for disrupting cells (such as a French Press or a Beat Beater apparatus sold by BioSpec Products Inc., Bartlesville, Okla.), and sonicating the samples (e.g., for 1-10 cycles, or more), under conditions effective to lyse at least a majority of the remaining cells (e.g., most or all of the remaining the cells). The order and number of these steps, and the particular conditions in which they are carried out, will vary according to what bacterium is used, and how difficult it is to lyse the bacterium. The precise conditions can be determined routinely by a skilled worker, without undue experimentation. The inventors investigated a number of combinations of steps for lysing cells and preparing DNA and found, unexpectedly, that this particular combination of steps was the by far the most efficient, especially for preparing DNA from cells that are difficult to lyse, such as Gram-positive bacteria.

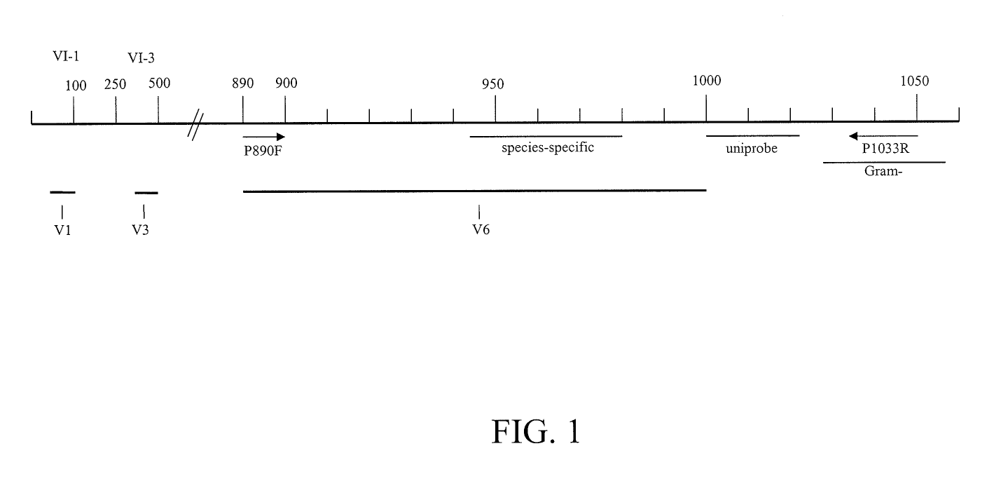

Some of the methods of the invention involve the use of PCR primers that prime virtually universally across species of eubacterial 16S rRNA genes. For example, the amplicon that the primers amplify can comprise a first conserved region that is emblematic of eubacteria, a second conserved region that differs between Gram-positive and Gram-negative eubacteria, and a particular (first) divergent region, if a eubacterial 16S rRNA gene is present in a PCR reaction. See FIG. 1 for a diagrammatic representation of these and other portions of the eubacterial rRNA gene that are discussed herein. Note that although the primers indicated in the figure “flank” a region of interest, the sequences of the primers become part of the amplicon. Therefore, the “flanking” primers also contain some of the Gram-specific sequences.

Such primers are virtually universal in applicability across the eubacteria. Thus, the primers amplify a segment of 16S rRNA genes of other eubacteria that also has the structure of containing the two highly conserved regions and the divergent region. Therefore, the primers employed will amplify a segment of S. aureus 16S rRNA in the presence of S. aureus DNA template. But they will amplify virtually any other eubacterial 16S rRNA in the presence of that eubacterial DNA template. Exemplary primers are discussed herein, and are indicated as SEQ ID NO:5 and SEQ ID NO:6. Other primers having similar functional properties can also be used. These can be readily designed by conventional methods, such as inspection of known sequences of 16S rRNA genes or by use of computer programs such as ClustalW from the European Bioinformatics Institute (world wide web site ebi.ac.uk/clustalw.htm).

In general, the primers of the present invention are defined in terms of their relationship to S. aureus 16S rRNA. However, as discussed above, other primers having similar properties can be used. A preferred amplicon, which contains the two conserved regions and the divergent region, bracketed by two conserved regions for primer binding, preferably contains at least 100, 125, 150, 160, or 170 bp. A larger amplicon permits identification of more divergent regions which can be used to uniquely identify eubacterial species. A suitable segment of S. aureus 16S rRNA gene comprises nucleotides 890 to 1051, with reference to the Staphylococcus aureus sequence AF015929. A first conserved region of S. aureus 16S rRNA gene within this segment that can be utilized advantageously to identify the presence of a eubacterium comprises nucleotides 1002 to 1024 of S. aureus 16S rRNA gene. A divergent region within this segment that can be utilized to identify S. aureus comprises nucleotides 912-1002. In particular, the sequence from positions 945 to 978 can be used. The primers used in the Examples herein are p890F and p1033R (SEQ ID NO:5 and SEQ ID NO:6, respectively.)

Detection of the first conserved region permits the detection of eubacteria generically. The first conserved region of 16S rRNA genes which is detected in a method of the invention comprises at least 18 contiguous nucleotides that are at least 80% identical among at least 10 or at least 14 eubacterial species. The conserved regions can be at least 15, 20, 25, or 30 contiguous nucleotides. Preferably the regions are identical across a broad range of eubacterial species. However, divergence of up to 5, 10, 15, or 20% can be accommodated. The divergent regions comprise at least 10 contiguous nucleotides and differ by at least 3, 4, 5, or 6 nucleotides from a divergent region found in Bradyrhizobium japonicum 16S rRNA gene. See GenBank Accession Nos. D12781, X87272, and X71840. The divergent regions can comprise between 10 and about 30 contiguous nucleotides, and may be at least 15, 20, or 25 contiguous nucleotides.

In assays of the invention, different probes are used to hybridize to the first conserved region and to the divergent region of eubacteria. If any eubacteria are present, regardless of species, hybridization to the first conserved region will occur. However, hybridization may not occur to a divergent probe if the probe does not correspond to the species of eubacteria which is present. Multiple divergent probes may be used simultaneously in a single or multiple real-time PCR reactions to identify a particular species.

Detection of a particular divergent region by a method of the invention permits the identification of a particular species of eubacteria. A comparison of the sequences of this divergent region of 15 species of eubacteria is shown in FIG. 5 of U.S. patent publication 2004/0235010. Probes that are specific for sequences from this region in other bacterial species have also been designed by the inventors and shown to work effectively in a method of the invention. Such probes were designed based on 16S rRNA sequence data obtained from GenBank and aligned with sequences from a variety of other bacterial species using the program ClustalW. As part of the design process, the primers and probe sequences were analyzed against all known published genetic sequences in the GeneBank database to determine the degree of similarity using the softward program NCBI BLAST (Basic Local and Alignment Search Tool). Among the many suitable probes for sequences of this divergent region are probes for a number of biothreat (BT) bacteria. These include, e.g., Bacillus anthracis (5′ CCTCTGACAACCCTAGAGATAGGGCTTCTC 3′ (SEQ ID NO:15)), Yersinia pestis (5′ CACAGAATTTGGCAGAGATGCTAAAGTGCC 3′(SEQ ID NO:16)), and Francisella tularensis (5′ CGAACTTTCTAGAGATAGATTGGTGCTTCGGAA3′(SEQ ID NO:17)). A variety of other eubacterial pathogen-specific probes have been designed for this variable region and shown to be effective in a method of the invention. These include, e.g., Listeria monocytogenes—5′AAGGGAAAGCTCTGTCTCCAGAGTGGTCAA3′ (SEQ ID NO:18), Streptococcus agalactiae—5′ TGCTCCGAAGAGAAA GCCTATCTCTAGGCC 3′ (SEQ ID NO:19), Staphylococcus epidermidis—5′ AAAACTCTATCTCTAGAGGGGCTAGA GGATGTCAAG 3′ (SEQ ID NO:20), Streptococcus pneumoniae—5′ TCACCTCTG TCCCGAAGGAAAACTCTATCTCTAGA 3′ (SEQ ID NO:21), Escherichia coli—5′ ACATTCTCATCTCTGAAAACTTCCGTGGATGTC 3′ (SEQ ID NO:22), Haemophilus influenza—5′ AAGGCACAAGCTCATCTCTGAGCTCTTCTTAGG 3′ (SEQ ID NO:23), and Neisseria meningitis—5′ CACTCCTCCGTCTCCGGAGGATTCC 3′ (SEQ ID NO:24).

The number of probes for this divergent region that are used in an assay of the invention depends, for example, on the number of etiologic agents that might be responsible for particular condition with which a patient presents. For SA joint disease, for example, a panel of probes for 5-6 potentially etiologic bacteria is generally sufficient. For bacterial meningitis, a different panel, of about 7 probes, is generally sufficient. The number of probes that can be assayed simultaneously in a single RT-PCR reaction is limited only by the number of spectrally distinguishable fluorogenic probe dyes that are currently available. Generally, a subject (patient) presenting with a particular condition is infected with only a single type of pathogenic bacteria. However, even if several organisms are present in a patient, one can assay simultaneously for several organisms. The different fluorogenic probes will not interfere with one another in the assay.

Detection of the second conserved region permits the determination if a eubacterium is Gram-positive or Gram-negative. Table 7 displays long conserved sequences found in 16S rRNA DNA from a variety of Gram-positive or Gram-negative bacteria. Of particular interest in this table are the two short, highly conserved sequences, SEQ ID NO:1 and SEQ ID NO:2. The bold, underlined residues in these sequences are particularly important for distinguishing Gram-positive from Gram-negative bacteria. Therefore, fluorogenic probes for performing Gram-typing by a method of the invention should contain one or both of these important residues. The smallest probe that can function effectively in such an assay is probably about 3 or 5 nts in length. Other probes can have, for example, 8, 10, 12, 14, 16, 18, 20, 22, 24, 26, 28, 30 or more nucleotides, or more, provided that the residues lie within the conserved portions of the sequences shown in Table 7. Probes of the invention can contain substitutions, small insertion or deletions, or other variations, provided that such a variant probe is at least about 80% (e.g., 85%, 87%, 90% or 95%) identical to the relevant sequence of the region as shown in Table 7.

Real-time polymerase chain reaction (RT-PCR) is employed in many of the assays described herein. RT-PCR is well-known in the art and can be practiced generally according to the known methods. See for example, Heid et al. (1996) Genome Res. 6, 986-994. Briefly, the assay is designed such that the labeling moieties on the 5′ and/or 3′ ends of each probe do not fluoresce unless PCR amplification of the sequence to which the probe binds has occurred, followed by hybridization of the probe to the amplified sequences, in which case fluorescence of the probe can be seen. The labeling moieties on both ends of a probe are fluorescent molecules, which quench one another. For simplicity, the labeling moiety on one end (e.g., the 5′ end) is sometimes referred to herein as a “fluorophore,” and the labeling moiety on the other end (e.g., the 3′ end) as a “quencher.” When a single stranded probe is not hybridized to a target and is free in solution, the probe molecule is flexible and folds back partially on itself, so that the quencher and the fluorophore are close together; the quencher thus prevents the probe from fluorescing. Furthermore, when a probe of the invention is hybridized to a single-stranded target, the two labeling moieties are close enough to one another to quench each other. However, without wishing to be bound by any particular mechanism, it is suggested that when the probe is hybridized to its target to form a perfect double stranded DNA molecule, a 5′ to 3′ exonuclease which recognizes perfect hybrids, and which is an activity of the enzyme used for PCR, cleaves the duplex, releasing the fluorophore. The fluorophore is thus separated from the quencher, and will fluoresce. The amount of detected fluorescence is proportional to the amount of amplified DNA.

In a real time PCR, the released fluorescent emission is measured continuously during the exponential phase of the PCR amplification reaction. Since the exponential accumulation of the fluorescent signal directly reflects the exponential accumulation of the PCR amplification product, this reaction is monitored in real time (“real time PCR”). Oligonucleotides used as amplification primers (e.g., DNA, RNA, PNA, LNA, or derivatives thereof) preferably do not have self-complementary sequences or have complementary sequences at their 3′ end (to prevent primer-dimer formation). Preferably, the primers have a GC content of about 50% and may contain restriction sites to facilitate cloning. Amplification primers can be between about 10 and about 100 nt in length. They are generally at least about 15 nucleotides (e.g., at least about 15, 20, or 25 nt), but may range from about 10 to a full-length sequence, and not longer than 50 nt. In some circumstances and conditions, shorter or longer lengths can be used. Amplification primers can be purchased commercially from a variety of sources, or can be chemically synthesized, using conventional procedures. Some exemplary PCR primers are described elsewhere herein.

Probes and conditions are selected, using routine conventional procedures, to insure that hybridization of a probe to a sequence of interest is specific. A probe that is “specific for” a nucleic acid sequence (e.g., in a DNA molecule) contains sequences that are substantially similar to (e.g., hybridize under conditions of high stringency to) sequences in one of the strands of the nucleic acid. By hybridizing “specifically” is meant herein that the two components (the target DNA and the probe) bind selectively to each other and not generally to other components unintended for binding to the subject components. The parameters required to achieve specific binding can be determined routinely, using conventional methods in the art. A probe that binds (hybridize) specifically to a target of interest does not necessarily have to be completely complementary to it. For example, a probe can be at least about 95% identical to the target, provided that the probe binds specifically to the target under defined hybridization conditions, such a conditions of high stringency.

As used herein, “conditions of high stringency” or “high stringent hybridization conditions” means any conditions in which hybridization will occur when there is at least about 85%, e.g., 90%, 95%, or 97 to 100%, nucleotide complementarity (identity) between a nucleic acid of interest and a probe. Generally, high stringency conditions are selected to be about 5° C. to 20° C. lower than the thermal melting point (Tₘ) for the specific sequence at a defined ionic strength and pH. Appropriate high stringent hybridization conditions include, e.g., hybridization in a buffer such as, for example, 6× SSPE-T (0.9 M NaCl, 60 mM NaH₂ PO₄, 6 mM EDTA and 0.05% Triton X-100) for between about 10 minutes and about at least 3 hours (in one embodiment, at least about 15 minutes) at a temperature ranging from about 4° C. to about 37° C.). In one embodiment, hybridization under high stringent conditions is carried out in 5×SSC, 50% deionized Formamide, 0.1% SDS at 42° C. overnight.

Methods for labeling probes with fluorophores are conventional and well-known. Suitable fluorescer-quencher dye sets will be evident to the skilled worker. Some examples are described, e.g., in Holland et al. (1991) Proc. Natl. Acad. Sci. 88, 7276-7280; WO 95/21266; Lee et al. (1993) Nucleic Acids Research 21, 3761-3766; Livak et al. (1995), supra; U.S. Pat. No. 4,855,225 (Fung et cd); U.S. Pat. No. 5,188,934 (Menchen et al.); PCT/US90/05565 (Bergot et al.), and others. Suitable fluorophores include rhodamine dyes and fluorescein dyes, including, e.g., fluorescein; 6-carboxyfluorescein (FAM™) 2′,4′,5′,7′,-tetrachloro-4,7-dichlorofluorescein (HEX™) 2′,7′-dimethoxy-4′,5′-6-carboxyrhodamine (JOE™), N′,N′,N′,N′-tetramethyl-6-carboxyrhodamine (TAMRA™) and 6-carboxy-X-rhodamine (ROX™). Other dyes which can be used include TET™; VIC™; Texas Red®, Cy3™, Cy5™, SYBR®Green I, NED™, CAL Fluor Orange 560, BHQ-1, and others. Various combinations of fluorophores can be used. Suitable pairings include, e.g., FAM™/ROX™; FAM™/SYBR® Green I; VIC®/JOE™; NED™/TAMRA™/ROX HEX™ FAM™/SYBR® Green I; VIC®/JOE™; NED™/TAMRA™/Cy3™; ROX™/Texas Red®; Cy5™ dyes; and CAL Fluor Orange 560/BHQ-1. These and other suitable dyes are available commercially, e.g. from Invitrogen (Carlsbad, Calif.), Applied Biosystems (Foster City, Calif.), Biosearch Technologies (Novato, Calif.), and others.

If two or more fluorogenic probes are used in a single RT-PCR, dyes on the probes preferably have non-overlapping emission spectra. Thus, their signals can be interpreted unambiguously as representing hybridization and/or amplification of a particular probe without further testing. As the technology advances and the number of suitable fluorescer-quencher dye sets increases, the number of probes that can be used in a single RT-PCR will increase. When two or more probes are used in a single reaction, it is sometimes beneficial to design the probes to anneal to opposite strands of the template DNA.

The fluorogenic probes described in the Examples herein function by means of FRET (fluorescence resonance energy transfer). The FRET technique utilizes molecules having a combination of fluorescent labels which, when in proximity to one another, allows for the transfer of energy between labels. See, e.g., the Examples herein or “iQ5 Real Time PCR Detection System” Manual (Bio-Rad, Hercules, Calif.). Other well-known methods for the detection of real-time PCR will be evident to a skilled worker. For example, molecular beacons can be used.

Methods of PCR amplification, and reagents used therein, as well as methods for detecting emission spectra, are conventional. For guidance concerning PCR reactions, see, e.g., PCR Protocols: A Guide to Methods and Applications (Innis et al. eds, Academic Press Inc. San Diego, Calif. (1990)). These and other molecular biology methods used in methods of the invention are well-known in the art and are described, e.g., in Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & sons, New York, N.Y. Hybridization as used according to the present invention, refers to hybridization under standard conditions used for real-time PCR to achieve amplification.

Suitable PCR controls will be evident to a skilled worker. Some typical controls are described in the Examples herein. In some embodiments of the invention, it may desirable to compare the level of a signal to a baseline value. For example, in an assay to detect a particular species of eubacterium, the baseline value can be the amount of signal expected in a sample that does not comprise the species being assayed. If a normalization control is used, the baseline value can be from a database of values in which the species being assayed is not present. Positive controls can be utilized in a similar fashion. It may be desirable to express the results of an assay in terms of a statistically significant increase in signal compared to a baseline value. A “significant” increase or decrease in the amount of signal when a DNA of interest is present in a sample, as used herein, can refer to a difference which is reproducible or statistically significant, as determined using statistical methods that are appropriate and well-known in the art, generally with a probability value of less than five percent chance of the change being due to random variation. Some such statistical tests will be evident to a skilled worker. For example, a significant increase in the amount of signal compared to a baseline value can be at least about 50% higher (e.g., at least about 2-fold, 5-fold, 10-fold, or more higher). In another embodiment of the invention, when Gram typing is assayed, one can determine the ratio of signal when a Gram-positive and a Gram-negative probe is used in the assay.

A “melt curve” assay of the invention is carried out by PCR amplifying sequences from at least 3 (e.g., 3, 4, 5, 6 or even more) hypervariable regions of the 16S rRNA gene in bacteria that are flanked by highly conserved regions, and then subjecting the resulting amplicons to high resolution melt curve profile analysis. The Example illustrating this method amplifies DNA from regions V1, V3 and V6, using primers that bind to the conserved regions on either side of each hypervariable region. One or more of the remaining six hypervariable regions characterized by Chakravorty et al. (2007) (supra) can be used instead of or in addition to these three hypervariable regions. Other regions that can be used include, e.g., hypervariable regions within the 23S rRNA gene of eubacteria, which also comprises hypervariable regions flanked by highly conserved regions, or segments from the intragenic region between the 16S and 23S genes.

A melt curve assay of the invention can be performed in conjunction with a method to determine if a eubacterium is present in a sample. For example, a sample can first be subjected to an RT-PCR assay in which a suitable segment of DNA is PCR amplified in the presence of a labeled Uniprobe. Only samples that are determined to be positive for the presence of a eubacterium are then analyzed by the melt curve procedure. In another embodiment, an assay to determine if eubacterial DNA is present in a sample is carried by assaying for the presence of eubacterial DNA as the amplicons from the at least three hypervariable regions are generated. When PCR reactions are carried out to generate amplicons for subsequent melt analysis, the PCRs are carried out in the presence of an intercalating dye such as LC Green Dye (as shown in Example III herein). The dye is useful for detecting melted DNA, since the dye is released as the DNA melts, leading to an increase in the signal as the DNA melts. This dye can also be used to confirm the presence of bacterial DNA in a sample. If the PCR reactions to generate the amplicons are carried out in a spectrophotometer, one can monitor whether there is eubacterial DNA by looking for a change in fluorescence or a signal accompanying the intercalation of the dye into the double stranded DNA as it is produced. Only samples in which eubacterial DNA is shown to be present by this method are subsequently subjected to melt curve analysis.

By a “sample” (e.g. a test sample) from a subject is meant a sample that may have (is suspected of having) a eubacterial infection. The sample can be from any of a variety of subjects including, e.g., a variety of vertebrates, such as laboratory animals (e.g., mouse, rat, rabbit, monkey, or guinea pig), farm animals (e.g., cattle, horses, pigs, sheep, goats, etc.), and domestic animals or pets (e.g., cats or dogs). Non-human primates and, preferably, humans, are included. Among the types of samples that can be tested by a method of the invention are, e.g., blood, urine, saliva, tears, sweat, cerebrospinal fluid (CSF), lymph fluid, serum, plasma, joint fluid, peritoneal fluid, or pleural fluid. The source of sample will be a function of the bacterial species to be identified. For example, synovial samples are appropriate for detecting etiologic agents of septic arthritis, and CSF samples are appropriate for assaying for etiologic agents for bacterial meningitis. In one embodiment of the invention, the samples are clinical samples that are easy to obtain and easy to store. Particularly suitable samples are those from patients who are suspected due to clinical findings of having bacteremia.

Methods for obtaining samples and preparing them for analysis are conventional and well-known in the art. Samples can be treated by a variety of methods to lyse cells in a sample and to liberate DNA from them. A particularly useful procedure for preparing DNA samples is described elsewhere herein. This procedure which comprises steps of (a) concentrating cells in the sample, for example by centrifugation, (b) resuspending the pelleted cells in a suitable diluent, such as decontaminated molecular grade water, (c) incubating the resuspended cells with Lysostaphin and Proteinase K and (d) subjecting the enzyme treated samples to one or more cycles of freezing and thawing, or to another mechanical method for disrupting cells, and sonication. This procedure is particularly useful for non-complex preparations, such as, e.g., urine, spinal fluid, joint fluid, or peritoneal fluid. The procedure cannot be used for complex preparations such as blood, which comprise genomic DNA in, for example, T-cells, or for samples that are not sterile, such as stool or sputum, and which thus contain many other types of potentially contaminating bacteria.

A useful method for removing bacterial DNA that may be undesired contaminants of reagents or vessels is to use a filtration step. Preferably the filtration of the reagents will remove double-stranded DNA contaminants having a length of at least 125 bp. U.S. patent application 2004/0235010 describes such a filtration procedure. An alternative decontamination step can employ restriction endonuclease digestion of unwanted contaminating DNA. Care must be taken to ensure that the primers and probes are not susceptible to digestion by the restriction endonuclease employed. Preferably a site for digestion will be found within the amplicon but not within the primers themselves. Thus all components of the reaction mixture, excluding the test sample, can be treated with the restriction endonuclease. The restriction endonuclease is subsequently inactivated to prevent destruction of analyte in the test sample.

An assay method of the invention can be carried out in conjunction with other analytic methods to determine if a sample comprises a eubacterium, if it is Gram-positive or Gram-negative, or to genotype the bacterium. Such methods include, for example, conventional culture based methods, DNA sequencing, mass spectrometry, direct probe hybridization, a variety of nucleic acid amplification methods, DNA microarrays, etc.

One aspect of the invention is a kit for detecting whether a sample contains a eubacterial infection or contamination, comprising one or more agents for detecting the presence of a eubacterium, determining whether it is Gram-positive or Gram-negative, and/or determining what species is present. The agents in the kit can encompass, e.g., primers for PCR amplification, fluorogenic probes of the invention, agents to conduct high resolution melt curve analysis, or the like. The kit may also include additional agents suitable for detecting, measuring and/or quantitating the amount of PCR amplification or for generating high resolution melt curve profiles. A skilled worker will recognize components of kits suitable for carrying out a method of the invention. In addition to the clinical uses discussed herein, kits of the invention can be used for experimental applications.

Optionally, a kit of the invention may comprise instructions for performing the method. Optional elements of a kit of the invention include suitable buffers, containers, or packaging materials. The reagents of the kit may be in containers in which the reagents are stable, e.g., in lyophilized form or stabilized liquids. The reagents may also be in single use form, e.g., for the performance of an assay for a single subject.

In the foregoing and in the following examples, all temperatures are set forth in uncorrected degrees Celsius; and, unless otherwise indicated, all parts and percentages are by weight.

### EXAMPLES

### Example I

### Rapid PCR-Based Diagnosis of Septic Arthritis by Early Gram-Type Classification and Species Identification

### A. Materials and Methods

### 1. Bacterial Species and Mock-Up Samples

Thirty six clinically relevant bacterial organisms and DNA, including the six most common SA-related organisms, were obtained from American Type Culture Collection (ATCC, Manassas, Va.) or the Johns Hopkins Hospital (JHH) clinical laboratory (Division of Medical Microbiology, Johns Hopkins School of Medicine, Baltimore, Md.) (Table 1).

A single isolated colony of each organism was inoculated in Tryptic Soy Broth (TSB, Beckton and Dickinson, Sparks, Md.) and incubated at 37° C. overnight. For LOD (limit of detection) determination, serial dilutions of each SA related organisms were spiked into culture-negative and DNA free synovial fluid samples. These mockup samples were processed based on the protocol (“Extraction of DNA”) described below. LOD was calculated based on colony forming units per milliliter (CFU/ml). DNA was also extracted from our panel of clinically relevant organisms for testing the analytical specificity of our Gram positive and Gram negative probe, as well as all pathogen-specific probes, as shown in Table 2.

2. Clinical Samples and Study Location

Clinical synovial fluid samples were derived from patients who presented with suspected acute SA to one of three clinical sites (the Emergency Department [ED], the Orthopedic Clinic or the Rheumatology Clinic), of the Johns Hopkins University Hospital and The Johns Hopkins Bayview Medical Center, both large tertiary care hospitals, from July 2006 to July 2007. One hundred twenty-one samples were obtained from the microbiology laboratories and were provided for research as ‘excess’, deidentified specimens after the microbiology laboratories had performed standard microbiologic testing including cultivation. The study was approved by The Johns Hopkins Institutional Review Board.

‘Excess’ samples were processed as follows: (1) samples were given a random study number and taken from the microbiology laboratory to the research laboratory where they were stored at −20° C. for later DNA extraction and PCR analysis; (2) a database which included the microbiology accession number and the random study number was created; (3) the microbiology database was queried for culture results; (4) the database was deidentified; and (5) samples were analyzed by PCR; and (6) PCR results were compared with microbiology culture results.

### 3. Extraction of DNA

Synovial fluid samples were thawed at room temperature and diluted with molecular grade water (Roche diagnostics, Basel, Switzerland) to reduce sample viscosity, in sample: water ratios of 1:10, 1:100, 1:500, and 1:1000 for a final volume of 500 μl. Each 500 μl sample aliquot was centrifuged at 3,200×g for 10 minutes in Eppendorf-5415 D centrifuge (Westbury, N.Y.) and the pellet was resuspended in 50 μl of molecular grade water. A 10 μl mixture of 1×(0.32 μg/μl) Lysozyme (Sigma Aldrich, Saint louis, Mo.) and 1×(0.5 μg/μl) Lysostaphin (Sigma Aldrich) was then added to the sample and incubated at 37° C. for 15 minutes. One microliter aliquot of 1× Proteinase K (MAGNA LC Kit-I, Roche Diagnostics, Indianapolis, Ind.) was added and the sample was incubated at 65° C. for 10 minutes. Samples were subjected to a freeze-thaw cycle for 10 minutes at −80° C. and 5 minutes at 95° C. Samples were then sonicated in a Bransonic T9000 (Shelton, Conn.) for 10 minutes before undergoing PCR testing.

### 4. Design of Primers and Probes

The target site within the 16S rRNA gene (which encompasses the hypervariable V6 region) and design of conserved primers (p891F and p1033R—SEQ ID NOs:5 and 6, respectively) and probe (Uniprobe—SEQ ID NO:7) were as previously described (Yang et al. (2002) J Clin Microbiol. 40, 3449-5414). Gram-typing and SA related pathogen-specific probe sequences are shown in Table 1. These probes were designed based on 16S rRNA sequence data obtained from GenBank and aligned with sequences from various clinically relevant bacterial species using the program ClustalW at the world wide web site, ebi.ac.uk/clustalw.htm. Theoretical specificity of all designed primer and probe sequences were further analyzed using NCBI's BLAST (Basic Local Alignment Search Tool).

TABLE 1
Probe sequences

| Organism | Probe sequences |
|---|---|
| Universal Gram | FAM 5′ AGGTGGTGCATGGTTGTCGTCAGC 3′ MGB (SEQ ID NO: 3) |
| Positive | |
| Staphylococcus | FAM 5′ CCTTTGACAACTCTAGAGATAGAGCCTTCCC 3′ MGB |
| aureus | (SEQ ID NO: 8) |
| Staphylococcus | TET 5′ AAAACTCTATCTCTAGAGGGGCTAGAGGATGTCAAG 3′ MGB |
| epidermidis | (SEQ ID NO: 20) |
| Streptococcus | TET 5′ TCACCTCTGTCCCGAAGGAAAACTCTATCTCTAGA 3′ MGB |
| pneumoniae | (SEQ ID NO: 21) |
| Streptococcus | FAM 5′ TGCTCCGAAGAGAAAGCCTATCTCTAGGCC 3′ MGB (SEQ ID |
| agalactiae | NO: 19) |
| Universal Gram | VIC 5′ ACAGGTGCTGCATGGCTGTCGTCAGCT 3′ MGB (SEQ ID NO: 4) |
| Negative | |
| Escherichia coli | FAM 5′ ACATTCTCATCTCTGAAAACTTCCGTGGATGTC 3′ MGB |
| | (SEQ ID NO: 22) |
| Neisseria | FAM 5′ TCTCCGGAGGATTCCGCACATGTCAAAA 3′ MGB (SEQ ID |
| gonorrhoea | NO: 56) |
| Uniprobe | VIC 5′ CACGAGCTGACGACARCCATGCA 3′ MGB (SEQ ID NO: 7) |

### 5. PCR Master Mix Preparation

Each PCR reaction was performed in 50 μl total volume, which consisted of 30 μl of PCR master mix and 20 μl of sample input. PCR master mix contained 25 μl of 2× Taqman Universal PCR Mix (PE Applied Biosystems, Foster city, Calif.), 1.5 μl of 67 μM forward primer and reverse primer. The 2× Taqman Universal PCR Mix and the primers underwent an ultra filtration step using Microcon YM-1000 centrifugal filter device (Millipore Corporation, Bedford, Mass.) by centrifuging at 3,200×g for 10 minutes to remove potential exogenous background DNA contamination. Following ultra filtration, an additional 1 ul of 2.5 units of Amplitaq Gold LD (PE Applied Biosystems, Foster city, Calif.) and 1 μl of 10 μM probe were added to make up the final master mix before sample was added. PCR was then performed using ABI 7900 HT Sequence Detection System (P.E Applied Biosystem, Foster city, Calif.). The cycling conditions used were: Pre-incubation at 50° C. for 2 min, Denaturation at 95° C. for 10 min and 40 repeats at 95° C. for 15 sec, annealing/extension temperature at 60° C. for 60 sec.

### 6. Positive, Negative, and Exogenous Internal Positive Control Preparation

Ultra pure water was used as non-template PCR negative control (NTC). Culture negative synovial fluids were screened using our universal probe PCR assay. Samples with a C_{T} value (see “Post PCR analysis”) equal to or higher than NTC controls were pooled and established for use as a standard negative control. An exogenous internal positive control (IPC, PE Applied Biosystems, Foster City, Calif.) was used on all clinical samples according to manufacturer's instructions in order to rule out sample inhibition to PCR.

### 7. PCR Assay Algorithm

Clinical synovial fluid samples were tested for the presence of eubacteria using Uniprobe PCR. Positive samples by the Uniprobe PCR were further analyzed with parallel PCRs using both Gram-positive and Gram-negative probes, as well as our panel of pathogen-specific probes.

### 8. Post PCR Analysis

Amplification data were analyzed by the SDS software (PE—Applied Biosystems), which calculates AR, using the equation Rₙ (+)−Rₙ (−). Rₙ (+) is the emission intensity of the reports divided by the emission intensity of the quencher at any given time, whereas Rₙ (−) is the value of Rₙ (+) prior to amplification. Thus, ΔRₙ indicates the magnitude of the signal generated. The threshold cycle, or C_{T}, is the cycle at which statistically significant increase in ΔRn is first detected. The C_{T} is inversely proportional to the starting amount of target DNA. Amplification plots were generated by plotting ΔRn versus C_{T}.

All clinical samples, standardized pooled negative control, and 1PC controls were performed in triplicates. The average and standard deviation for the pooled negative control replicates from each run were calculated. Due to the potential for day-to-day inter-run variability, the cutoff C_{T} value for each run was defined as 3 standard deviations above the negative control average, as described, e.g., in Bobo et al. (1991) Lancet 338, 847-50. Any sample with a C_{T} value higher than the cutoff value was considered PCR negative and vice versa. All samples with discordant findings between PCR and culture results were plated on 5% sheep blood agar plates (Becton, Dickinson and Company) to assess for bacterial growth. Any samples with growth on agar were sent to the JHH clinical microbiology laboratory for identification. Amplified PCR products from discordant samples were sequenced in-house by Johns Hopkins University CORE Genetics facility.

Accuracy of Uniprobe PCR was determined by the observed clinical sensitivity and specificity as compared to conventional culture results. 95% confidence intervals for clinical sensitivity and specificity were estimated by exact binominal test method.

### B. Results

### 1. LOD and Analytical Specificity

The LOD of our Uniprobe PCR was determined by testing mock-up synovial fluid samples containing serially diluted organisms most commonly found in SA. As shown in Table 2, our Uniprobe PCR demonstrated high analytical sensitivity with LOD of 10¹-10² CFU/ml.

TABLE 2
LOD of Uniprobe PCR in CFU/ml

| Organisms | CFU/ml |
|---|---|
| Strpetococcus pneumoniae | 20 |
| ATCC # 49619 | |
| Streptococcus agalactiae | 50 |
| ATCC # 13813 | |
| Staphylococcus aureus | 20 |
| ATCC # 25923 | |
| Staphylococcus epidermidis | 110 |
| ATCC # 12228 | |
| Escherichia coli | 40 |
| ATCC # 25922 | |

We also evaluated the analytic specificity of our Gram-typing probes and our select panel of pathogen-specific probes by testing against DNA extracted from 36 clinically relevant bacterial organisms. All Gram-typing probes and pathogen-specific probes correctly identified their respective target organisms (Table 3).

TABLE 3
Cross reactivity of Gram-type and Pathogen Specific Probes

| Organism | GP | GN | ESCO | NEGO | STAG | STAU | STEP | STPN |
|---|---|---|---|---|---|---|---|---|
| Acinetobacter sp. | X | ✓ | X | X | X | X | X | X |
| Bacteroides fragilis | X | ✓ | X | X | X | X | X | X |
| Bacillus anthracis | ✓ | X | X | X | X | X | X | X |
| Bacillus cereus | ✓ | X | X | X | X | X | X | X |
| Bordetella pertussis | X | ✓ | X | X | X | X | X | X |
| Brucella ovis | X | ✓ | X | X | X | X | X | X |
| Campylobacter jejuni | X | ✓ | X | X | X | X | X | X |
| Citrobacter freundii | X | ✓ | X | X | X | X | X | X |
| Corneybacterium sp. | ✓ | X | X | X | X | X | X | X |
| Coxiella burnetti | X | ✓ | X | X | X | X | X | X |
| Escherichia coli | X | ✓ | ✓ | X | X | X | X | X |
| Francisella phylomeragia | X | ✓ | X | X | X | X | X | X |
| Francisella tularensis | X | ✓ | X | X | X | X | X | X |
| Group B Streptococcus | ✓ | X | X | X | ✓ | X | X | X |
| Haemophilus influenzae | X | ✓ | X | X | X | X | X | X |
| Helicobacter pylori | X | ✓ | X | X | X | X | X | X |
| Klebsiella pneumoniae | X | ✓ | X | X | X | X | X | X |
| Legionella pneumophila | X | ✓ | X | X | X | X | X | X |
| Listeria monocytogenes | ✓ | X | X | X | X | X | X | X |
| Micrococcus sp. | ✓ | X | X | X | X | X | X | X |
| Neisseria meningititis | X | ✓ | X | X | X | X | X | X |
| Neisseria gonnorhoeae | X | X | X | ✓ | X | X | X | X |
| Pseudomonas aeruginosa | X | ✓ | X | X | X | X | X | X |
| Proteus mirabilis | X | ✓ | X | X | X | X | X | X |
| Proteus vulgaris | X | ✓ | X | X | X | X | X | X |
| Salmonella sp. | X | ✓ | X | X | X | X | X | X |
| Serratia marscens | X | ✓ | X | X | X | X | X | X |
| Staphylococcus aureus | ✓ | X | X | X | X | ✓ | X | X |
| Staphylococcus epidermidis | ✓ | X | X | X | X | X | ✓ | X |
| Streptococcus faecalis | ✓ | X | X | X | X | X | X | X |
| Streptococcus pneumoniae | ✓ | X | X | X | X | X | X | ✓ |
| Streptococcus pyogenes | ✓ | X | X | X | X | X | X | X |
| viridans group stre ptococci | ✓ | X | X | X | X | X | X | X |
| Treponema pallidum | X | ✓ | X | X | X | X | X | X |
| Yersinia pestis | X | ✓ | X | X | X | X | X | X |
| Yersinia pseudotuberculosis | X | ✓ | X | X | X | X | X | X |

GP—Gram positive,
GN—Gram negative,
STAU—S. aureus,
STEP—S. epidermidis,
STAG—S. agalactiae,
STPN—S. pneumoniae,
ESCO—E. coli,
NEGO—N. gonorrohoeae,

### 2. Clinical Sensitivity and Specificity

One hundred and twenty one clinical synovial fluid samples were collected from patients with suspected SA and tested using our PCR assay. Among the samples collected, 21 were culture positive and 100 were culture negative. As shown in Table 4, 20 of 21 culture-positive samples tested positive by Uniprobe PCR, and 97 of 100 culture-negative samples tested negative. The calculated clinical sensitivity and specificity of Uniprobe PCR are 95.2% (95% CI: 76.2-99.9%) and 97.0% (95% CI: 91.5-99.4%), respectively. The percent agreement of Uniprobe PCR with culture was 96.7% (95% CI: 91.8-99.1%).

TABLE 4
Clinical Joint Fluid Samples: Uniprobe PCR Vs Culture Results

| | Culture | | |
|---|---|---|---|
| Uniprobe | (+) | (−) | Total |
| (+) | 20 | 03* | 23 |
| (−) | 1* | 97 | 98 |
| Total | 21 | 100 | 121 |

*Please refer to Table 6 for further analyses of the discordant results.

Of the 20 Uniprobe-positive samples, our Gram-positive and Gram-negative probes were in complete concordance with culture results (Table 5); our pathogen-specific probes were concordant with culture results in 16/20 samples. (13 Staphylococcus aureus, 2 Staphylococcus epidermidis, and 1 Group B Streptococcus). The 97 samples which tested negative by Uniprobe PCR all had positive Internal Positive Control (IPC) results, indicating that there was no PCR inhibitor present.

TABLE 5
Clinical Synovial Fluid Samples: Uniprobe & Type Specific PCR—Concordant results

| Sample no | Culture results | PCR results | Uni | GP | GN | STAU | STEP | STAG | STPN | ESCO | NEGO |
|---|---|---|---|---|---|---|---|---|---|---|---|
| BAY-051 | STAU | STAU | ✓ | ✓ | X | ✓ | X | X | X | X | X |
| BAY-062 | STAU | STAU | ✓ | ✓ | X | ✓ | X | X | X | X | X |
| BAY-133 | STAU | STAU | ✓ | ✓ | X | ✓ | X | X | X | X | X |
| BAY-160 | STAU | STAU | ✓ | ✓ | X | ✓ | X | X | X | X | X |
| BTW-J0003 | STAU | STAU | ✓ | ✓ | X | ✓ | X | X | X | X | X |
| BTW-J0026 | STAU | STAU | ✓ | ✓ | X | ✓ | X | X | X | X | X |
| BTW-J0039 | STAU | STAU | ✓ | ✓ | X | ✓ | X | X | X | X | X |
| BTW-J0077 | STAU | STAU | ✓ | ✓ | X | ✓ | X | X | X | X | X |
| BTW-J0078 | STAU | STAU | ✓ | ✓ | X | ✓ | X | X | X | X | X |
| BTW-J0084 | STAU | STAU | ✓ | ✓ | X | ✓ | X | X | X | X | X |
| BTW-J0094 | STAU | STAU | ✓ | ✓ | X | ✓ | X | X | X | X | X |
| BTW-J0096 | STAU | STAU | ✓ | ✓ | X | ✓ | X | X | X | X | X |
| BTW-J0115 | STAU | STAU | ✓ | ✓ | X | ✓ | X | X | X | X | X |
| BTW-J0079 | STEP | STEP | ✓ | ✓ | X | X | ✓ | X | X | X | X |
| BTW-J0098 | STEP | STEP | ✓ | ✓ | X | X | ✓ | X | X | X | X |
| BTW-J0102 | STAG | STAG | ✓ | ✓ | X | X | X | ✓ | X | X | X |

Uni—Uniprobe,
GP—Gram positive,
GN—Gram negative,
STAU—S. aureus,
STEP—S. epidermidis,
STAG—S. agalactiae,
STPN—S. pneumoniae,
ESCO—E. coli,
NEGO—N. gonorrohoeae,

Discordant Uniprobe PCR

Four samples showed discordant results between culture and Uniprobe PCR. Three (BTW-J0049, BTW-J0086, and BTW-J0101) were reported negative by culture but were positive by Uniprobe PCR (Table 6). Repeat culturing of these samples did not show any growth after 3 days of incubation. Sequencing of the PCR product did not yield quality data for organism identification. One sample (BTW-J0019) was reported culture positive for S. epidermidis (grew only after 2 days) but negative by Uniprobe PCR. The sample was reported to grow only in culture broth but not by conventional plating. Repeat culturing of the sample revealed no growth after 3 days of incubation. Sequencing of the PCR product did not yield organism-specific quality data.

TABLE 6
Clinical Synovial Fluid Samples: Uniprobe & Type Specific PCR—discordant results

| Sample no | Culture results | PCR results | Uni | GP | GN | STAU | STEP | STAG | STPN | ESCO | NEGO | Comments |
|---|---|---|---|---|---|---|---|---|---|---|---|---|
| BTW-J0069 | STAU | STEP | ✓ | ✓ | X | X | ✓ | X | X | X | X | Sequenced—no quality data |
| BTW-J0019 | STAU | Negative | X | X | X | X | X | X | X | X | X | Grew in broth after 3 days |
| BTW-J0030 | †VGS | S. pneumo | ✓ | ✓ | X | X | X | X | ✓ | X | X | Sequencing—S. pneumo |
| BTW-J0031 | †VGS | S. pneumo | ✓ | ✓ | X | X | X | X | ✓ | X | X | Sequencing—S. pneumo |
| BAY-157 | *Group G | No probe | ✓ | ✓ | X | X | X | X | X | X | X | Sequencing—Group G |
| BTW-J0049 | Negative | STEP | ✓ | N/A | N/A | X | ✓ | X | X | X | X | Sequenced—no quality data |
| BTW-J0086 | Negative | Positive | ✓ | N/A | N/A | X | X | X | X | X | X | Sequenced—no quality data |
| BTW-J0101 | Negative | Positive | ✓ | N/A | N/A | X | X | X | X | X | X | Sequenced—no quality data |

Uni—Uniprobe,
GP—Gram positive,
GN—Gram negative,
STAU—S. aureus,
STEP—S. epidermidls,
STAG—S. agalactiae,
STPN—S. pneumoniae,
ESCO—E. coli,
NEGO—N. gonorrohoeae,
VGS—viridans group streptococci
*BAY-157 was reported as Group B, it was replated, recultured and identified as Group G. Sequencing confirmed as Group G
†BTW-J0030 was reported as S. viridans, replated with no growth at 3 days. Sequencing confirmed as S. pneumoniae

Discordant Pathogen-Specific PCRs

Four of the 20 Uniprobe positive, culture positive samples had discordant results between conventional microbiological methods and our pathogen-specific PCR. One sample (BTW-J0069) was identified as S. aureus by culture, but tested positive only by our S. epidermidis probe. Sequencing studies showed no organism-specific quality data. Two samples (BTW-J0030, BTW-J0031) reported as viridans group streptococci by culture were positive by our Streptococcus pneumoniae probe. No viridans group streptococci probe was available for testing, but sequencing results from both of these samples confirmed S. pneumoniae, consistent with our PCR finding. Both samples were re-cultured but there was no growth after 3 days. One sample (BAY-157) which was reported as group B streptococcus (S. agalactiae) by culture was group B streptococcus PCR probe negative. Repeat culturing of this sample identified a group G streptococcus. Sequencing of the amplicon from this sample also confirmed a group G streptococcus. Both the sequencing results and the re-culture results were concordant with our PCR probe negative result, but discordant with the initial culture findings (we do not have a Group G streptococcus probe).

### 3. Assay Performance Time

Time from sample collection to PCR results including DNA extraction (60 minutes) and PCR amplification and detection (120 minutes) or a total assay performance time of 180 minutes.

C. Discussion

We observed the presence of either PCR inhibitors or excess DNA, as evidenced by negative internal positive controls from some highly viscous joint fluid samples, which required pre-dilution before testing positive by PCR. Thus, samples were routinely tested at several dilutions as mentioned in the methods.

The high specificity of the RT-PCR assay of the invention is likely attributable, at least in part, to the methods we employed for minimizing exogenous eubacterial DNA contamination, which can be pronounced in broad-based 16S rRNA eubacterial PCR assays. In addition to stringent adherence to standard precautionary measures for reducing carryover DNA, we employed our previously reported decontamination measure using size-based ultrafiltration to reduce contaminating eubacterial DNA from PCR reagents, primers, and DNA polymerase prior to amplification (See, e.g., Yang et al. (2002) J Clin Microbiol. 40, 3449-54 and U.S. Patent Application 2004/0234010). The new procedure for preparing DNA samples which is disclosed herein also required minimal sample transfer between steps, further decreasing risk of contamination. Finally, samples were scored as positive by Uniprobe only if their C_{T} values were at least 3 standard deviations from the non-template controls, minimizing the likelihood of false positives (Bobo et al. (1991) (supra)). Use of a third ‘resolver’ test (e.g. PCR targeting an alternative pathogen-specific gene) may help adjudicate these discrepant cases in future analyses, and identify an unusual etiologic agent not included in our panel of “most common” SA causing organisms.

Early identification and characterization by Gram-type of a suspected pathogen detected by Uniprobe PCR can allow for more focused selection of antimicrobial therapy and can ultimately contribute to both decreased incidence of adverse drug effects and reduction of emergence of multi-drug resistant pathogens. Our Gram-type specific probes demonstrated 100% specificity in both the test panel of organisms and all of the culture-positive clinical samples. Moreover, BLAST search against the GenBank database under the most stringent criteria confirmed 100% Gram-specificity (data not shown). Our panel of six pathogen-specific probes was selected to detect the majority (˜80%) of etiologic agents responsible for SA (Dubost et al. (2002) Ann Rheum Dis. 61, 267-269). Despite potential sequence homology between closely related species within the target hypervariable region of the 16S rRNA gene, each of our probes showed high specificity at the species level. Three of 4 cases (BTW-J0030, BTW-30031, BAY-157) which were culture and specific probe discordant we likely identified on initial conventional microbiological evaluation, since sequencing of the amplified product confirmed our probe findings. Further, in at least one case, superior specificity of our genotyping approach over traditional culture based phenotypic methods for species identification was demonstrated, (i.e. sample initially labeled as group B streptococcus by the hospital laboratory, identified by our multiprobe assay as followed by sequencing as a group G streptococcus, and ultimately also confirmed by repeat culture as a group G streptococcus).

Minimizing the number of processing steps for sample preparation together with use of real-time PCR chemistry methods provides an assay which is rapid and relatively simple as compared to traditional culture or PCR methods. The complete process (from specimen collection to target detection) can be achieved in less than 2 hours with use of the most up-to-date high-speed thermocyclers. In theory, as the technology improves, the assay can be carried out even more rapidly. The two-step PCR algorithm (first Uniprobe PCR; followed if positive by Gram-specific and appropriate panels of pathogen-specific PCRs) offers potential cost-savings as it reduces unnecessary PCR testing associated with Uniprobe negative cases or Gram-positive or negative reactives. As technology improves, and differential detection of multiple fluorescent probe labels becomes available, it should be possible to achieve a multiplex detection platform for all probe sequences in a single PCR reaction. The use of molecular beacons for direct enzyme-independent amplicon hybridization, (as well as melting curve analysis of the amplicons) can provide a time and cost saving improvement of this assay.

In conclusion, our findings provide proof-of-concept that a real-time multiprobe eubacterial PCR assay can diagnose SA with speed and accuracy. The clinical applicability of our assay algorithm as a “molecular triage tool” in the ED can extend beyond SA detection. Prompt recognition and characterization of systemic bacterial infections in otherwise “sterile body fluids” in acutely ill febrile patients would be invaluable in routine clinical care, leading to early directed therapy, reduced unnecessary hospitalizations, and potentially decreased rates of antimicrobial resistance. We envision this PCR assay will ultimately serve as an adjunct to, rather than a replacement for, conventional culture methodologies, which will still be required for confirmation and susceptibility testing.

### Example II

### Design of Gram-Negative and Gram-Positive Probes

To design probes that can distinguish between Gram positive and Gram negative bacteria, we aligned partial 16S rRNA sequences from within the universal PCR target region of various clinically relevant bacterial pathogens. The comparisons are shown in Table 7, below. In addition to SEQ ID NO:1 and SEQ ID NO:2, the sequences shown in table have SEQ ID NOs: 25 to 55, reading from top to bottom of the table.

| | | | |
|---|---|---|---|
| Gram-Negative Sequence | | TGCTGCATGGCTGT (SEQ ID NO: 2) | |
| Acinetobacter sp | | TTCGGG--AACTTACATACAGGTGCTGCATGGCTGTCGTCAGCTCGTGT | |
| pseudomonas_sp | | TTCGGG--AACTCTGACACAGGTGCTGCATGGTTGTCGTCAGCTCGTGT | |
| Aeromonas_caviae | | TTCGGG--AATCAGAACACAGGTGCTGCATGGCTGTCGTCAGCTCGTGT | |
| Aeromonas_hydrophila | | TTCGGG--AATCAGAACACAGGTGCTGCATGGCTGTCGTCAGCTCGTGT | |
| Klebsiella pneumoniae | | TTCGGG--AACTGTGAGACAGGTGCTGCATGGCTGTCGTCAGCTCGTGT | |
| Serratia_marcescens | | TTCGGG--AACTCTGAGACAGGTGCTGCATGGCTOTCGTCAGCTCGTGT | |
| Enterobacter | | TTCGGG--AACTCTGAGACAGGTGCTGCATGGCTGTCGTCAGCTCGTGT | |
| Escherichia coli | 1009 | TTCGGG--AACCGTGAGACAGGTGCTGCATGGCTGTCGTCAGCTCGTGT | 1058 |
| Citrobacter_freundii | | TTCGGG--AACTCTGAGACAGGTGCTGCATGGCNGTCGTCNGCTCGTGT | |
| Salmonella sp | | TTCGGG--AACTGTGAGACAGGTGCTGCATGGCTGTCGTCAGCTCGTGT | |
| Yersinia pestis | | TTCGGG--AACTGTGAGACAGGTGCTGCATGGCTGTCGTCAGCTCGTGT | |
| Morganella_morganii | | TTCGGG--AACTCTGAGACAGGTGCTGCATGGCTGTCGTCAGCTCGTGT | |
| Proteus sp | | TTCGGG--AACGCTGAGACAGGTGCTGCATGGCTGTCGTCAGCTCGTGT | |
| Haemophilus influenzae | | TTCGGG--AACTTAGAGACAGGTGCTGCATGGCTGTCGTCAGCTCGTGT | |
| Legionella pneumophila | | TTCGGG--AACACTGATACAGGTGCTGCATGGCTGTCGTCAGCTCGTGT | |
| Neisseria meningitidis | | TTCGGG--AGCCGTAACACAGGTGCTGCATGGCTGTCGTCAGCTCGTGT | |
| Stenotrophomonas_sp | | TTCGGG--AACGCGAACACAGGTGCTGCATGGCTGTCGTCAGCTCGTGT | |
| Francisella tularensis | | TC--GG--AACGCAGTGACAG-TGCTGCACGGCTGTCGTCAGCTCGTGT | |
| Brucella sp | | GTTCGGCTGGATCGGAGACAGGTGCTGCATGGCTGTCGTCAGCTCGTGT | |
| Mycobacterium sp | | TCCCTTGTGGCCTGTGTGCAGGTGGTGCATGGCTGTCGTCAGCTCGTGT | |
| Campylobacter jejuni | | CTTGCTAGAACTTAGAGACAGGTGCTGCACGGCTGTCGTCAGCTCGTGT | |
| Mycoplasma pneumoniae | | ---AGGTTAACCGAGTGACAGGTGGTGCATGGTTGTCGTCAGCTCGTGT | |
| Gram-Positive MB Sequence | | TGGTGCATGGTTGT (SEQ ID NO: 1) | |
| Propionibacterium sp | | TCTTTTGGGGTTGGTTCACAGGTGGTGCATGGCTGTCGTCAGCTCGTGT | |
| Corynebacterium sp | | TCCCTTGTGGCTCACATACAGGTGGTGCATGGTTGTCNTCAGCTCGTGT | |
| Enterococcus sp | | CTTCGGGGG-CAAAGTGACAGGTGGTGCATGGTTGTCGTCAGCTCGTGT | |
| Staphylococcus aureus | | CTTCGGGGGACAAAGTGACAGGTGGTGCATGGTTGTCGTCAGCTCGTGT | |
| Bacillus sp | | CTTATGGGACAGCGGTGACAGGTGGTGCATGGTTGTCGTCAGCTCGTGT | |
| Streptococcus pneumoniae | | NCTTCGGGACAGAGGNGACAGGTGGNGCATNGTNGTCGTCAGCTCGTGT | |
| Viridans Streptococcus | | ACTTCGGTACATCGGTGACAGGTGGTGCATGGTTGTCGTCAGCTCGTGT | |
| Clostridium sp | | CCTTCGGGGACAGGGAGACAGGTGGTGCATGGTTGTCGTCAGCTCGTGT | |
| Peptostreptococcus sp | | TCTTCGGAGACTGCTAAACAGGTGGTGCATGGTTGTCGTCAGCTCGTGT | |

### Example III

### Rapid Identification of Class A Biothreat (“BT”) and Other Clinically Relevant Bacterial Species Using Universal PCR Coupled with High Resolution Melt Curve Profile Analysis

The inventors previously used the previously Uniprobe/species-specific RT-PCR assay to perform BT-surveillance and detection, using pathogen-specific TaqMan probes that were designed for Category A bacterial agents (Yang et al. (2008) Acted Emerg Med. 15, 388-9213). The assay demonstrated high analytical sensitivity, but was limited by inability to differentiate closely related pathogens due to decreased specificity of the TaqMan probe chemistry and high sequence homology within selected hypervariable region of the 16S rRNA gene. Probe-based amplicon characterization accordingly limits screening to a finite number of anticipated pathogens. Alternative strategies for amplicon analysis, such as sequencing and mass-spectrometry, allow broader scale product characterization but are costly, time-consuming, and lacking in throughput. High-resolution melt analysis (Witter et al. (2003) Clin Chem. 49, 853-6010) offers a simple low cost, closed-tube approach to amplicon analysis and can be easily integrated with PCR. We report here a strategy for rapid, highly specific identification of BT and non-BT related bacterial pathogens which couples eubacterial PCR with high-resolution melt analysis.

Three hypervariable regions (V1, V3, V6), each flanked by highly conserved sequences within the 16S rRNA gene, were selected for primer design (Chakravorty et al. (2007) J Microbiol Methods 69, 330-93). The particular endpoints of these regions will vary in different bacterial organisms, but a skilled worker can readily determine the precise location of regions in bacteria of interest, e.g., by inspecting known sequences of the rRNA genes. In this study, sequence data for clinically or BT relevant bacteria were obtained from GenBank and aligned using ClustalW (as described above) to determine sequence variability. Primer pairs used to target hypervariable regions were: V1-F (5′-GYGGCG NACGGGTGAGTAA-3′) (SEQ ID NO:9); V1-R (5′-TTACCYYACCAACTAGC-3′) (SEQ ID NO:10); V3-F (5′-CCA GACTCCTACGGGAGGCAG-3′) (SEQ ID NO:11); V3-R (5′-CGTATTACCGCGGCTGCTG-3′) (SEQ ID NO:12); V6-F (5′-TGGAGCATGTGGTTTAATTCGA-3′) (SEQ ID NO:13); and V6-R (5′-AGCTGACGACARCCATGCA-3′) (SEQ ID NO:14). All primers were analyzed using Integrated DNA Technology's online tool (at the world wide web site idtdna.com) to minimize formation of primer dimerization.

One hundred and seven common, BT-related, and BT-surrogate organisms, composed of 58 different bacterial species of either American Type Culture Collection (ATCC) strains, clinical isolates, inactivated or non-pathogenic strains, were used for analysis. (Table 8).

TABLE 8

| Non-BT related organisms | v1 | v3 | v6 |
|---|---|---|---|
| Acinetobacter sp. ATCC 5459 | b | b | a |
| Acinetobacter calcoaceticus | a | d | a |
| Aerococcus viridans | f | h | c |
| Bacteriodes fragilis^{a} | b | a | e |
| Bordetella pertussis^{a} | c | c | f |
| Bordetella parapertussis | b | c | h |
| Campylobacter jejunii^{a} | c | a | e |
| Clostridium difficile | g | f | a |
| Clostridium perfringens | a | d | d |
| Corneybacterium sp^{a} | c | c | e |
| Coxiella brunetti^{c} | d | b | g |
| Coxiella brunette strain “9 mile” | d | b | g |
| Chlamydia pneumoniae^{a} | g | c | a |
| Chlamydia trachomatis^{a} | f | a | b |
| Citrobacter freumdii^{a} | a | c | a |
| Enterobacter aerogenes | c | b | a |
| Enterococcus gallinarum | i | i | h |
| Enterococcus faecium | a | a | e |
| Enterobacter faecalis ATCC 29212 | i | i | a |
| Escherichia coli ATCC 25927 | e | d | c |
| Helicobacter pylori^{a} | g | b | a |
| Haemophilus influenzae ATCC 49247 | a | g | d |
| Klebsiella pneumoniae^{a} | h | c | a |
| Legionella pneumophila ATCC 33495 | b | a | b |
| Listeria monocytogenes ATCC 7648 | a | e | a |
| Micrococcus sp. ATCC 14396 | a | b | b |
| Mooraxella catarrhalis | h | i | d |
| Mycobacterium kansasii | i | c | a |
| Mycobacterium gordonae | d | i | i |
| Mycobacterium fortuitum | b | i | b |
| Mycoplasma pneumoniae^{a} | a | d | g |
| Mycoplasma hominis^{a} | b | b | e |
| Neisseria meningitis ATCC 6250 | d | f | c |
| Neisseria gonorrhoeae^{a} | b | c | a |
| Oligella urethralis | a | a | i |
| Pasteurella multocida | a | i | a |
| Pseudomonas aeruginosa ATCC 10145 | a | b | c |
| Propionibacterium acnes | e | i | e |
| Proteus mirabilis^{a} | a | a | f |
| Proteus vulgaris^{a} | c | a | i |
| Salmonella sp. ATCC 31194 | c | e | a |
| Serratia marscecens ATCC 8101 | a | j | c |
| Staphylococcus aureus ATCC 25923 | b | b | h |
| Staphylococcus epidermidis ATCC 12228 | b | a | h |
| Staphylococcus lugdunensis | g | i | i |
| Staphylococcus sapropyticus | h | i | h |
| Streptococcus pneumoniae ATCC 49619 | g | d | g |
| Streptococcus pyogenes^{a} | a | e | b |
| Streptococcus agalactiae ATCC 13813 | a | e | d |
| Treponema pallidum^{a} | f | b | e |
| Viridans Group Streptococci ATCC 10556 | c | e | f |
| Yersinia enterolitica | d | i | a |
| Category A BT agents | V1 | V3 | V6 |
| Bacillus anthracis^{d} | c | a | a |
| Bacillus anthracis 3001 | c | a | a |
| Bacillus cereus^{a} | a | a | d |
| Bacillus cereus strain BC 9634 | a | a | d |
| Bacillus cereus strain BC 12480 | a | a | d |
| Bacillus cereus strain BC 27877 | a | a | d |
| Bacillus cereus strain BC 7064 | a | a | d |
| Bacillus cereus strain BC B33 | a | a | d |
| Bacillus cereus strain BC 1410-1 | a | a | d |
| Bacillus cereus strain BC 1410-2 | a | a | d |
| Bacillus cereus strain BC T | a | a | d |
| Bacillus cereus strain BC 2599 | a | a | d |
| Bacillus cereus strain BC 2464 | a | a | d |
| Bacillus cereus strain BC 7687 | a | a | d |
| Bacillus cereus strain BC 10329 | a | a | d |
| Bacillus cereus strain BC 11143 | a | a | d |
| Bacillus cereus strain BC 11145 | a | a | d |
| Bacillus cereus strain BC 1414 | a | a | d |
| Bacillus cereus strain BC 7089 | a | a | d |
| Bacillus cereus strain BC 6464 | a | a | d |
| Bacillus cereus strain BC 6474 | a | a | d |
| Bacillus cereus strain BC 7004 | a | a | d |
| Bacillus cereus strain BC 10987 | a | a | d |
| Bacillus cereus strain BC 23674 | a | a | d |
| Bacillus cereus strain BC 9189 | a | a | d |
| Bacillus cereus strain BC 246 | a | a | d |
| Bacillus cereus strain BC 13472 | a | a | d |
| Bacillus subtilis 110 NA | a | a | g |
| Bacillus subtilis strain SB168 | a | a | g |
| Bacillus subtilis strain W168 | a | a | g |
| Bacillus subtilis strain W23 | a | a | g |
| Bacillus subtilis strain her 148 | a | a | g |
| Bacillus subtilis strain T6 | a | a | g |
| Bacillus subtilis strain ATCC 27505 | a | a | g |
| Bacillus subtilis strain ATCC 15841 | a | a | g |
| Franscicella phylomiragia (GAO1-2810)^{e} | a | g | g |
| Franscicella tularensis (LVSB)^{f} | b | h | g |
| Franscicella tularensis Fran 0001 | b | h | g |
| Franscicella novacida Fran 7002 | b | h | g |
| Yersinia pseudotuberculosis (PB1/+)^{g} | a | g | c |
| Yersinia pseudotuberculosis strain Schulze's | a | g | c |
| group type B/ATCC 6903 | | | |
| Yersinia pseudotuberculosis strain Schulze | a | g | c |
| group II/ATCC 27802 | | | |
| Yersinia pseudotuberculosis strain CDC | a | g | c |
| P62/ATCC 29910 | | | |
| Yersinia pseudotuberculosis strain Schulze's | a | g | c |
| group III/ATCC 13980 | | | |
| Yersinia pseudotuberculosis strain raffinose | a | g | c |
| positive ATCC 4284 | | | |
| Yersinia pseudotuberculosis strain ATCC | a | g | c |
| 13979 | | | |
| Yersinia enterolitica strain 0: 9 Serotype | a | g | d |
| Yersinia enterolitica strain WA.C | a | g | d |
| Yersinia kristtensenii strain ATCC 336640 | a | g | c |
| Yersinia kristtensenii strain CDC 1458-51 | a | g | c |
| Yersinia ruckerii strain isolated from Fish | a | g | c |
| kidney | | | |
| Yersinia ruckerii strain ATCC 33644 | a | g | c |
| Yersinia fredericksenii | a | g | c |
| Yersinia pestis (P14-)^{h} | a | b | d |
| Yersinia pestis strain 1122 | a | b | d |

Table 8: Each species has a unique three letter grouping code. The unique grouping codes allow for differentiation and identification between these 42 non-BT and BT-related bacterial pathogens.
^{a}Clinical isolates
^{b}Brucella ovis DNA obtained from Joany Jackman, PhD, Applied Physics Laboratory, Johns Hopkins University, Baltimore, MD.
^{c}Coxiella brunettei DNA from Steven Dumbler, MD, Department of Pathology, School of Medicine, Johns Hopkins University, Baltimore, MD.
^{d}Inactivated non-pathogenic strain.
^{e}Non pathogenic strain obtained from Centre for Disease Control and Prevention, Fort Collins, Colorado, via Walter Reed Army Medical Hospital, Washington, D.C.
^{f}LVSB—Live vaccine strain type.
^{g}Wild type strain.
^{h}De-pigmented and virulence pCD1-negative.

Ten to fifteen colonies of each bacterial organism were inoculated in 200 μl of molecular grade water (Roche Molecular Diagnostics, Indianapolis) and DNA was extracted using Roche MAGNA Pure instrument. DNA from 9 clinical synovial fluid samples with positive culture results were collected and processed as in Yang et al. (2008) J. Clin, Microbial. 46, 1386-90.

Extracted DNA from each organism or clinical sample was subjected to 3 PCR reactions, each targeting the V1, V3, V6 hypervariable regions, respectively. Every PCR reaction was performed in 10 μl total volume, comprised of 8 μl PCR master mix and 2 μl of target input. PCR master mix contained 4 μl 2× Universal PCR Mix (Idaho Technology, Salt lake city, Utah) and LC green dye for high resolution melting. 1.0 μl of 1.5 μM forward primer and reverse primer was added to the master mix. Each PCR reaction contained one primer set. PCR was performed using Rapid Cycler (RC-2; Idaho technology, Salt Lake city, Utah). Cycling conditions: Denaturation at 95° C. for 30 sec. followed by 45 cycle repeats of: 95° C. for 30 sec, and annealing/extension at 60 C/72° C. for 60 sec, one cycle of: 95° C. for 30 sec, and 28 C for 30 sec.

Each post-PCR sample was subjected to high resolution melt analysis on the HR-1 Lightscanner instrument (Idaho technology, Salt Lake City, Utah). Melting conditions were 60° C. to 95° C. Data acquisitions were done for every 0.1° C. increase in temperature. Melt profiles for each organism performed in triplicates and analyzed using Lightscanner software (Idaho technology). Melt analysis was subjected to fluorescence normalization and temperature shift to obtain the minimum inter- and intra-run variability.

Each of the 100 bacterial organisms tested has a derivative plot generated from HRMA for each of the analysis subsets (V1, V3, and V6) based on the primer set used (FIG. 2). Each derivative plot revealed a single dominant peak, which was absent in the non-template control, suggesting the presence of a single amplified sequence. The derivative plots have been demonstrated to be reproducible from run to run despite varying target DNA concentrations over a 10,000-fold range (data not shown). Using the derivative plot of Staphylococccus aureus as the reference, difference plots of the 100 tested organisms generated were compared within their analysis subset. Each difference plot was assigned a code letter and only plots with similar characteristics within the same analysis subset shared the same code letter. Although different species were observed to share similar plots within the same analysis subset, each species was associated with an unique “melt profile” of 3-letter code when all 3 analysis subsets were included. Even closely related species (e.g. Bacillus anthracis versus Bacillus cereus) with a single nucleotide difference within some of our target regions could be differentiated and correctly identified (FIG. 2). Identical melt profiles were observed among the various strains of the same species (Table 7). We also performed HRMA on Eubacterial PCR products derived from 9 clinical synovial fluid samples with positive culture results and the melt profiles generated were compared to our reference database of 58 different bacterial species for identification. The species identified based on melt profiles correctly matched those derived from the culture results in all 9 samples tested.

In this study, we demonstrate a simple, yet powerful approach to amplicon analysis for rapid bacterial species identification, and differentiation of BT agents from their related surrogates. This approach relies on eubacterial real-time PCR followed by high-resolution melt analysis.

Despite the high discriminatory precision of high resolution melt analysis, we found that amplicons of very different sequences may generate similar melt curves. One way to resolve such “melting groups” would be to perform subsequent heteroduplex-melt analyses between amplicons of unknown and reference bacterial species A potential drawback with this approach is that closely related species with identical sequences within the amplified region may not be readily differentiated. We chose to analyze the melt profiles based on three, instead of one of the 16S, hypervariable regions. This yielded a unique set of melt plots for every non-BT or BT-relevant bacterial organism tested, with even closely related species able to be discerned.

Future studies, using expanded panels of clinically relevant bacterial species, followed by clinical validation studies using samples from patients with suspected systemic bacterial infections, and animals that are infected with biothreat agents, are expected to confirm reproducibility and specificity of the assay method.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications (including provisional patent applications 61/011,522, filed Jan. 18, 2008; 61/011,529, filed Jan. 18, 2008; and 61/068,345, filed Mar. 6, 2008) cited above and in the figures are hereby incorporated in their entirety by reference.

1. A set of oligonucleotides for distimunshing Gram-positive eubacteria from Gram-negative eubacteria, wherein
(a) a first oligonucleotide, which is specific for Gram-positive eubacteria, consists of the sequence TGGTGCATGGTTGT (SEQ ID NO: 1)
or a variant thereof in which 1 or 2 of the residues are substituted with other nucleotides, provided that the G and T residues that are indicated with bold underlining are not altered;
or a variant of the oligonucleotide consisting of SEQ ID NO: 1 or of the substituted variant, which has up to 5 additional nucleotides at its 5′ end from the Propionibacter rRNA gene sequence shown in Table 7 and/or up to 13 additional nucleotides at its 3′ end from the Propionibacter rRNA gene sequence shown in Table 7;
and
(b) a second oligonucleotide, which is specific for Gram-negative eubacteria, consists of the sequence TGCTGCATGGCTGT (SEQ ID NO 2);
or a variant thereof in which 1 or 2 of the residues are substituted with other nucleotides, provided that the two C residues that are indicated with bold underlining are not altered;
or a variant of the oligonucleotide consisting of SEQ ID NO:2 or of the substituted variant which has up to 4 additional nucleotides at its 5′ end from the Acinetobacter rRNA gene sequence shown in Table 7 and/or up to 13 additional nucleotides at its 3′ end from the Acinetobacter rRNA gene sequence shown in Table 7.

2. The set of oligonucleotides of claim 1, wherein
(a) the first oligonucleotide, which is specific for Gram-positive eubacteria, consists of the sequence TGGTGCATGGTTGT (SEQ ID NO:1) and
(b) the second oliogonucletoide, which is specific for Gram-negative eubacteria, consists of the sequence TGCTGCATGGCTGT (SEQ ID NO:2)

3. The set of oligonucleotides of claim 1, wherein
(a) the first oligonucleotide, which is specific for Gram-positive eubacteria, consists of the sequence AGGTGGTGCATGGTTGTCGTCAGC (SEQ NO:3), or a variant thereof in which 1-3 of the residues are substituted with other nucleotides, provided that the G and T residues that are indicated with bold underlining are not altered; and
(b) the second oligonucleotide, which is specific for Gram-negative eubacteria, consists of the sequence ACAGGTGCTGCATGGCTGTCGTCAGCT (SEQ ID NO:4), or a variant thereof in which 1-3 of the residues are substituted with other nucleotides, provided that the two C residues that are indicated with bold underlining are not altered.

4. The set of oligonucleotides of claim 1, wherein
(a) the first oligonucleotide, which is specific for Gram-positive eubacteria, consists of the sequence AGGTGGTGCATGGTTGTCGTCAGC (SEQ ID NO:3), and
(b) the second oligonucleotide, which is specific for Gram-negative eubacteria, consists of the sequence ACAGGTGCTGCATGGCTGTCGTCAGCT (SEQ ID NO:4).

5. A method for detecting whether a eubacterium in a sample is Gram-positive or Gram-negative, comprising
(a) performing a real-time polymerase chain reaction (RT-PCR) using a sample which may comprise template DNA of the eubacterium, wherein the RT-PCR employs primers and at least two fluorogenic probes, each of which fluorogenic probe comprises a reporter dye and a quencher dye,
wherein the primers are complementary to two flanking regions of a S. aureus 16S rRNA gene, wherein the flanking regions flank a segment of the S. aureus 16S rRNA that comprises the sequence, or a complete complement thereof, of SEQ ID NO:3 or SEQ ID NO:4,
wherein a first of the at least two fluorogenic probes is complementary to the first oligonucleotide of claim 1, and is specific for Gram-positive eubacteria,
wherein the second of the at least two fluorogenic probes is complementary to the second oligonucleotide of claim 1, and is specific for Gram-negative eubacteria,
wherein the reporter dyes of the first and the second fluorogenic probes have non-overlapping, emission spectra; and
(b) monitoring fluorescence emissions of the reporter dyes;
wherein the detection of emissions characteristic of the reporter dye of the first probe indicates that the eubacterium is Gram-positive, and wherein the detection of emissions characteristic of the reporter dye of the second probe indicates that the eubacterium is Gram-negative.

6. A method for detecting a eubacterium, determining if the eubacterium is Gram-positive or Gram-negative, and determining the species of the eubacterium in a sample, comprising
(a) performing real-time polymerase chain reactions (RT-PCR) using one or more aliquots of a sample which may comprise template DNA of a first species of eubacteria, wherein each RT-PCR employs primers and at least one fluorogenic probe,
wherein the primers are complementary to two flanking regions of a S. aureus 16S rRNA gene, wherein the two flanking regions flank a segment of the S. aureus 16S rRNA gene comprising (1) a first conserved region, (2) a second conserved region which is diagnostic of Gram-positive or Gram-negative eubacteria; and (3) a first divergent region, and wherein
the first conserved region comprises at least 18 contiguous nucleotides that are at east 80% identical among at least 10 eubacterial species,
the second conserved region comprises the sequence SEQ ID NO:3 or SEQ ID NO:4, and
the first divergent region comprises at least 10 contiguous nucleotides and differs by at least 3 nucleotides from a second divergent region found in a Bradyrhizobium japonicum 16S rRNA gene;
wherein each of the fluorogenic probes comprises a reporter dye and a quencher dye, and wherein
(i) a first fluorogenic probe is complementary to the first conserved region of the S. aureus 16S rRNA gene;
(ii) a second fluorogenic probe is complementary to the first oligonucleotide of claim 1, and is specific for Gram-positive eubacteria, and
(iii) a third fluorogenic probe is complementary to the second oligonucleotide of claim 1, and is specific for Gram-negative eubacteria, and
(iv) a fourth fluorogenic probe is complementary to a third divergent region of the first species of eubacteria;
wherein between one and four of probes (i), (ii), (iii) and (iv) are present in each RT-PCR, and it two or more probes are present in a single RT-PCR, the reporter dyes of the two or more than probes have non-overlapping emission spectra;
(b) monitoring fluorescence emissions of the reporter dyes, wherein
detection of emissions characteristic of the reporter dye of the first probe indicates that a eubacterium is present in the sample,
detection of emissions characteristic of the reporter dye of the second probe indicates that the eubacterium is Gram-positive,
detection of emissions characteristic of the reporter dye of the third probe indicates that the eubacterium is Gram-negative,
detection of emissions characteristic of the reporter dye of the fourth probe indicates that the first species of eubacteria is present in the sample.

7. The method of claim 6, wherein a fifth fluorogenic probe is employed in the RT-PCR, wherein the fifth fluorogenic probe is complementary to a fourth divergent region of 16S rRNA gene in a second species of eubacteria; wherein the presence of the second species of eubacteria is determined when emissions characteristic of the dye on the fifth fluorogenic probe are detected.

8. The method of claim 6, wherein a sixth fluorogenic probe is employed in the RT-PCR, wherein the sixth fluorogenic probe is complementary to a fifth divergent region of 16S rRNA gene in a third species of eubacteria; wherein the presence of the third species of eubacteria is determined, when emissions characteristic of the dye on the sixth fluorogenic probe are detected.

9. The method of claim 6, wherein a seventh fluorogenic probe is employed in the RT-PCR, wherein the seventh fluorogenic probe is complementary to a sixth divergent region of 16S rRNA gene in a fourth species of eubacteria; wherein the presence of the fourth species of eubacteria is determined when emissions characteristic of the dye on the seventh fluorogenic probe are detected.

10. The method of claim 6, wherein
all four of the probes are present in a single RT-PCR, and the reporter dyes of each of the probes have non-overlapping emission spectra; or
a separate RT-PCR reaction is carried out in the presence of each of the four probes, which can have the same or different reporter dyes.

11. The method of claim 6, wherein the segment of S. aureus 16S rRNA gene comprises nucleotides 890 to 912 and 1033 to 1051 as shown in SEQ ID NO:5 and SEQ ID NO:6, respectively.

12. The method of claim 6, wherein the first conserved region of S. aureus 16S rRNA comprises nucleotides 1002 to 1024 as shown in SEQ ID NO:7.

13. The method of claim 6, wherein the first divergent region comprises nucleotides 912 to 1002 of S. aureus 16S rRNA.

14. The method of claim 6, wherein the sample is a synovial fluid sample.

15. The method of claim 14, wherein the synovial fluid, sample is from a subject suspected of having septic arthritis.

16. The method of claim 6, wherein the sample is blood, urine, saliva, tears, sweat, cerebrospinal fluid (CSF), lymph fluid, serum, plasma, joint fluid, peritoneal fluid, or pleural fluid.

17. The method of claim 6, wherein the DNA sample is prepared by a method consisting of
centrifuging the sample under conditions effective to pellet cells in the sample,
resuspending the pelleted cells in molecular grade water, which has been decontaminated from bacterial DNA by ultra-filtration,
incubating the resuspended cells with Lysostaphin and Proteinase K, under conditions effective to lyse cells and to degrade proteins in the cell,
subjecting the enzyme treated samples to one or more cycles of freezing and thawing, or to another mechanical method for disrupting cells, and sonicating the samples, under conditions effective to lyse at least a majority of the remaining cells.

18. The method of claim 6, wherein the components of the real-time PCR reaction mixture are filtered before the reaction begins to remove double stranded DNA components having a length of >125 bp to form a filtrate.

19. A kit, comprising a set of oligonucleotides of claim 1, and a pair of oligonucleotide primers for amplifying a segment of eubacterial 16S RNA gene of no more than 165 bp, wherein the amplified DNA comprises the sequence, or a complete complement thereof, of SEQ ID NO:3 or SEQ ID NO:4 and optionally, packaging materials or instructions for use of the kit.

20. The kit of claim 19, wherein the pair of oligonucleotide primers for amplifying the portion of the eubacterial 16S RNA gene consist of the sequence TGGAGCATGTGGTTTAATTCGA (SEQ ID NO:5) and TGCGGGACTTAACCCAACA (SEQ ID NO:6).

21. A method for determining the species of a eubacterium in a sample, comprising
(a) performing three RT-PCRs, using three aliquots of a sample which may contain template DNA of a eubacterium, wherein the size of the amplicons generated in each reaction is between 30 and 65 bp,
wherein the primers in the first PCR reaction amplify a sequence within hypervariable region V1 of a eukaryotic 16S rRNA gene, extending from nt 40-nt 201 (162 bp),
wherein the primers in the second PCR reaction amplify a sequence within hypervariable region V3 of the eukaryotic 16S rRNA gene, extending from nt 280-nt 484 (205 bp, and
wherein the primers in the first PCR reaction amplify a sequence within hypervariabe region V6 of the eukaryotic 16S rRNA gene, extending from nt 890-nt 1020 (131 bp);
(b) subjecting each of the three PCR-amplified DNA preparations to High Resolution Melting Analysis (HRMA), to generate a melt curve for each of the three amplified DNAs, and a melt profile signature taking into account all three melt curves; and
(c) comparing the melt profile signatures from each of the three PCR-amplified DNA preparations to a reference database of melt profile signatures for at least 60 bacterial species, as indicated in Table 7 and FIG. 2,
wherein if the melt profile signature for the sample is [substantially] the same as the melt profile signature of a known bacterial species, this indicates that the known bacterial species is present in the sample.

22. The method of claim 21, wherein
the PCR primers for the first PCR amplification are V1-F: 5′-GYGGCG NACGGGTGAGTAA-3′ (SEQ ID NO:9) and V1-R: 5′-TTACCYYACCAACTAGC-3′ (SEQ ID NO:10);
the PCR primers for the second PCR amplification are V3-F: 5′-CCA GACTCCTACGGGAGGCTG-3′ (SEQ ID NO:11) and V3-R: 5′-CGTATTACCGCGGCTGCAG-3′ (SEQ ID NO:12); and
the PCR primers for the third PCR amplification are V6-F: 5′-TGGAGCATGTGGTTTAATTCGA-3′ (SEQ ID NO:13) and V6-R: 5′-AGCTGACGACARCCATGCA-3′ (SEQ ID NO:14).

23. The method of claim 21, of wherein the sample is suspected of containing a clinically important bacterial pathogen or a Category A or B biothreat bacterial agent.

24. The method of claim 21, wherein the DNA sample is prepared by a method consisting of
centrifuging the sample under conditions effective to pellet cells in the sample,
resuspending the pelleted cells in molecular grade water, which has been decontaminated from bacterial DNA by ultra-filtration,
incubating the resuspended cells with Lysostaphin and Proteinase K, under conditions effective to lyse cells and to degrade proteins in the cell,
subjecting the enzyme treated samples to one or more cycles of freezing and thawing, or to another mechanical method for disrupting cells, and sonicating the samples, under conditions effective to lyse at least a majority of the remaining cells.

25. (canceled)

26. A method for detecting whether a eubacterium in a sample is Gram-positive or Gram-negative, comprising
(a) performing a real-time polymerase chain reaction (RT-PCR) using a sample which may comprise template DNA of the eubacterium, wherein the RT-PCR employs primers and at least two fluorogenic probes, each of which fluorogenic probe comprises a reporter dye and a quencher dye,
wherein the primers are complementary to two flanking regions of a S. aureus 16S rRNA gene, wherein the flanking regions flank a segment of the S. aureus 16S rRNA that comprises the sequence, or a complete complement thereof, of SEQ ID NO:3 or SEQ ID NO:4,
wherein a first of the at least two fluorogenic probes is complementary to the first oligonucleotide of claim 2, and is specific for Gram-positive eubacteria,
wherein the second of the at least two fluorogenic probes is complementary to the second oligonucleotide of claim 2, and is specific for Gram-negative eubacteria,
wherein the reporter dyes of the first and the second fluorogenic probes have non-overlapping emission spectra; and
(b) monitoring fluorescence emissions of the reporter dyes;
wherein the detection of emissions characteristic of the reporter dye of the first probe indicates that the eubacterium is Gram-positive, and wherein the detection of emissions characteristic of the reporter dye of the second probe indicates that the eubacterium is Gram-negative.

27. A method for detecting whether a eubacterium in a sample is Gram-positive or Gram-negative, comprising
(a) performing a real-time polymerase chain reaction (RT-PCR) using a sample which may comprise template DNA of the eubacterium, wherein the RT-PCR employs primers and at least two fluorogenic probes, each of which fluorogenic probe comprises a reporter dye and a quencher dye,
wherein the primers are complementary to two flanking regions of a S. aureus 16S rRNA gene, wherein the flanking regions flank a segment of the S. aureus 16S rRNA that comprises the sequence, or a complete complement thereof, of SEQ ID NO:3 or SEQ ID NO:4,
wherein a first of the at least two fluorogenic probes is complementary to the first oligonucleotide of claim 3, and is specific for Gram-positive eubacteria,
wherein the second of the at least two fluorogenic probes is complementary to the second oligonucleotide of claim 3, and is specific for Gram-negative eubacteria,
wherein the reporter dyes of the first and the second fluorogenic probes have non-overlapping emission spectra; and
(b) monitoring fluorescence emissions of the reporter dyes;
wherein the detection of emissions characteristic of the reporter dye of the first probe indicates that the eubacterium is Gram-positive, and wherein the detection of emissions characteristic of the reporter dye of the second probe indicates that the eubacterium is Gram-negative.

28. A method for detecting whether a eubacterium in a sample is Gram-positive or Gram-negative, comprising
(a) performing a real-time polymerase chain reaction (RT-PCR) using a sample which may comprise template DNA of the eubacterium, wherein the RT-PCR employs primers and at least two fluorogenic probes, each of which fluorogenic probe comprises a reporter dye and a quencher dye,
wherein the primers are complementary to two flanking regions of a S. aureus 16S rRNA gene, wherein the flanking regions flank a segment of the S. aureus 16S rRNA that comprises the sequence, or a complete complement thereof, of SEQ ID NO:3 or SEQ ID NO:4,
wherein a first of the at least two fluorogenic probes is complementary to the first oligonucleotide of claim 4, and is specific for Gram-positive eubacteria,
wherein the second of the at least two fluorogenic probes is complementary to the second oligonucleotide of claim 4, and is specific for Gram-negative eubacteria,
wherein the reporter dyes of the first and the second fluorogenic probes have non-overlapping emission spectra; and
(b) monitoring fluorescence emissions of the reporter dyes;
wherein the detection of emissions characteristic of the reporter dye of the first probe indicates that the eubacterium is Gram-positive, and wherein the detection of emissions characteristic of the reporter dye of the second probe indicates that the eubacterium is Gram-negative.

29. The method of claim 7, wherein a sixth fluorogenic probe is employed in the RT-PCR, wherein the sixth fluorogenic probe is complementary to a fifth divergent region of 16S rRNA gene in a third species of eubacteria, wherein the presence of the third species of eubacteria is determined when emissions characteristic of the dye on the sixth fluorogenic probe are detected.

30. The method of claim 7, wherein a seventh fluorogenic probe is employed in the RT-PCR, wherein the seventh fluorogenic probe is complementary to a sixth divergent region of 16S rRNA gene in a fourth species of eubacteria; wherein the presence of the fourth species of eubacteria is determined when emissions characteristic of the dye on the seventh fluorogenic probe are detected.

31. The method of claim 8, wherein a seventh fluorogenic probe is employed in the RT-PCR, wherein the seventh fluorogenic probe is complementary to a sixth divergent region of 16S rRNA gene in a fourth species of eubacteria; wherein the presence of the fourth species of eubacteria is determined when emissions characteristic of the dye on the seventh fluorogenic probe are detected.

32. The method of claim 22, wherein the sample is suspected of containing a clinical important bacterial pathogen or a Category A or B biothreat bacterial agent.
